(19) United States
(12) Patent Application Publication
(10) Pub. No.: US 2025/0046402 A1
(43) Pub. Date: Feb. 6, 2025

(54) UNIFIED MACHINE LEARNING FRAMEWORK TO EMULATE DENSITY FUNCTIONAL THEORY SIMULATIONS

(71) Applicant: Georgia Tech Research Corporation, Atlanta, GA (US)

(72) Inventors: Rampi Ramprasad, Atlanta, GA (US); Beatriz Gonzalez del Rio, Atlanta, GA (US)

(21) Appl. No.: 18/785,064

(22) Filed: Jul. 26, 2024

Related U.S. Application Data
(60) Provisional application No. 63/529,927, filed on Jul. 31, 2023.

(51) Int. Cl. G16C 20/30; G16C 20/70

(52) U.S. Cl. CPC G16C 20/30; G16C 20/70

(57) ABSTRACT

A method comprising providing a training data set to a machine learning (ML) system, the training data set indicative of density functional theory (DFT) data for a plurality of materials, the DFT data representing atomic configurations for the plurality of materials, determining a fingerprint for the atomic configuration for each of the plurality of materials, inputting the fingerprints into a machine learning system, generating, with the machine learning system, electron charge density data for the plurality of materials, inputting the electron charge density data and the fingerprint into a machine learning system, and predicting, with the machine learning system, one or more DFT properties of the plurality of materials.

### CROSS-REFERENCE TO RELATED APPLICATIONS

This application claims priority, and benefit under 35 USC § 119 (c), to U.S. Provisional Patent Application No. 63/529,927 filed 31-7-2023, the entire contents and substance of each are incorporated herein by reference in its entirety as if fully set forth below.

### STATEMENT REGARDING FEDERALLY SPONSORED RESEARCH OR DEVELOPMENT

This invention was made with government support under Agreement Nos. AWD-001075 and AWD-103067, awarded by the National Science Foundation. The government has certain rights in the invention.

### BACKGROUND OF THE DISCLOSURE

### 1. Field of the Invention

The various embodiments of the present disclosure relate generally to machine learning, and more particularly to using machine learning methods to emulate density functional theory.

### 2. Description of Related Art

Density functional theory (DFT) has become one of the most valuable computational tools for the materials research community. It has guided the discovery of new catalysts, the design of materials for energy storage, and the exploration of material behavior under extreme conditions, among other applications. The success of DFT lies in the transformation of the cumbersome many-electron many-nuclear problem of quantum mechanics to an effective one-electron Kohn-Sham (KS) equation. Solving the KS equation for a material with a given atomic configuration provides information about the ground state electronic structure of the system in the form of one-electron wave functions (or charge density) and one-electron eigenvalues (or density of states). These quantities, i.e., either the wave functions and eigenvalues, or the charge density and density of states, are the most essential and complete information of the material from which a host of properties can be computed such as the potential energy, atomic forces, and stress tensor. DFT-based research has seen several advancements over the last several decades in the areas of theory, algorithms, and computational infrastructure, instrumental in the above-mentioned discoveries. Nevertheless, practical and routine DFT calculations of complex materials involving several thousands of atoms to probe phenomena that occur over timescales of the order of nanoseconds or longer, remain inaccessible.

Over the last decade, machine learning (ML) based approaches are actively being considered in various ways to meet the length- and time-scale demands encountered during DFT computations. ML provides a powerful pathway to replace a cumbersome or expensive “input-output” problem with a cheap “surrogate” model. The accuracy and versatility of such models depend on the number and diversity of input-output examples the model has seen before, and the internal architecture of such models. The past decade has seen several successful ML efforts applied to various material properties and application spaces.

The present disclosure provides an efficient emulation of DFT by treating the KS equation itself as an input-output problem. Recent efforts have attempted to bypass explicitly solving the KS equation using a plane-wave basis representation of the electron density. While the accuracy of the model was demonstrated for small molecules, it was not transferable to large systems. Since this first attempt, two main methodologies have been investigated to predict the charge density: grid-based schemes and atom-based representations in terms of basis functions. The main advantage of a grid-based approach is the high accuracy obtained and general applicability to localized and delocalized electron densities. However, no information about individual atomic charges can be retrieved and the high computational cost can hinder the applicability to large databases. On the other hand, predicting the charge density as atomic contributions in terms of a basis set significantly reduces the computational cost and provides information on the individual atomic charges at the cost of lower accuracy, especially in systems with a delocalized electron density. One important advantage of atom-based representations is the higher transferability to new and larger systems, essential for a successful deployment of the model. Nevertheless, challenges still remain with respect to achieving comparable model performance for larger systems relative to smaller systems used during the training phase. Although methods have been developed to varying degrees of success to predict either the electronic structure or basic atomic properties such as total potential energy, atomic forces, or stress tensor, there is yet no scheme that has successfully unified simultaneous prediction of both types of properties in a comprehensive KS-DFT emulation.

### BRIEF SUMMARY OF THE INVENTION

An exemplary embodiment of the present disclosure provides a method, comprising providing a training data set to a machine learning (ML) system, the training data set indicative of density functional theory (DFT) data for a plurality of materials, the DFT data representing atomic configurations for the plurality of materials, determining a fingerprint for the atomic configuration for each of the plurality of materials, inputting the fingerprints into a ML system, generating, with the ML system, electron charge density data for the plurality of materials, inputting the electron charge density data and the fingerprint into the ML system, and generating, with the ML system, one or more DFT properties of the plurality of materials.

In any of the embodiments disclosed herein, the plurality of materials can comprise molecules, polymer chains, polymer crystal structures, or combinations thereof.

In any of the embodiments disclosed herein, the fingerprints can be indicative of a structural and/or chemical environment of each of the corresponding material.

In any of the embodiments disclosed herein, the electronic charge density data can comprise one or more Gaussian-type orbitals (GTOs).

In any of the embodiments disclosed herein, the method can further comprise projecting the one or more GTOs onto grid points.

In any of the embodiments disclosed herein, the one or more DFT properties can comprise one or more from the following list: potential energy, atomic force, stress tensor, density of states, valence band maximum, conduction band minimum, and bandgap.

In any of the embodiments disclosed herein, the plurality of materials can comprise a quantity of atoms, wherein a time of prediction for the ML system can be approximately linearly dependent on the quantity of atoms.

Another embodiment of the present disclosure provides a system comprising one or more processors that, individually or collectively, are configured to implement one or more steps of the various methods disclosed herein.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

Reference will now be made to the accompanying figures and diagrams, which are not necessarily drawn to scale, and wherein:

FIGS. 1A-1D: ML-DFT database and exemplary two-step workflow according to an exemplary embodiment of the present invention. FIG. 1A: The reference database contains DFT data from organic molecules, polymer chains, and polymer crystals. After creating the database, the atomic configurations are fingerprinted to describe the structural and chemical environment of each atom (FIG. 1B). Within the first step (FIG. 1C), the resulting atomic fingerprints are used as the input layer to predict the electronic charge density in terms of various Gaussian-type orbitals (GTOs) descriptors. The projection of these GTOs onto grid points provides the charge density. In the second step (FIG. 1D), the combined atomic fingerprints and charge density descriptors serve as input for the prediction of other DFT properties such as potential energy, atomic forces, stress tensor, density of states, valence band maximum, conduction band minimum, and the bandgap.

FIG. 2 is a schematic view of the internal reference system of carbon using its two nearest neighbors N₁ and N₂. The internal reference system of carbon is defined by {u₁, u₂, u₃}.

FIG. 3 is TABLE 2.

FIG. 4: Mean absolute percentage error (ϵ_{ρ}) of the charge density model for different methods. Both GTOs and STOs and various basis sets using only the hydrocarbon polymers were tested.

FIG. 5A: Histogram of the mean absolute percentage error, ϵ_{ρ}, for the charge density on the test configurations of the training and for new larger structures. The amount of structures in each bar is indicated as a percentage with respect to the total in each set. FIG. 5B: Charge density difference between DFT and ML-DFT for a molecule (cyclobutane), three polymer chains, and two crystalline polymers (Cryst).

FIGS. 6A-6E: Evaluation of atomic charges obtained from the charge density model. FIG. 6A: Parity plot between the DFT Hirshfeld charges and the ML-DFT atomic charges calculated using the charge density descriptors for the test configurations: triangles (carbon), circles (hydrogen), squares (nitrogen), and stars (oxygen). FIG. 6B: Histogram of the total charge error percentage on the test configurations. Atomic charges for some atoms in three different polymer chains obtained from (FIG. 6C) ML-DFT, (FIG. 6D) DFT Hirshfeld, and (FIG. 6E) DFT Bader method.

FIGS. 7A-7D: Density derived properties for the polymer chain [*]C(=O) NNC(=O)c1cnc([*])cn1. FIG. 7A: Charge density difference between DFT and ML-DFT, FIG. 7B: Reduced density gradient (RDG) plot and (FIG. 7C) noncovalent interactions isosurfaces at RDG=0.5.

FIG. 8: Error percentage of the predicted core electron density. Varying number of Is orbitals were tested for the description of the core electron density. Results shown for carbon (light color), nitrogen (medium light color), and oxygen (dark color).

FIGS. 9A-9B: Evaluation of predicted charge density on liquid Li. FIG. 9A: Charge density difference between DFT and ML-DFT for a test configuration of liquid Li at 500 K. FIG. 9B: Histogram of all the Li atom charges for all test configurations.

FIGS. 10A-10B: Explained variance of the first ten PCA. FIG. 10A: Histogram using only the atomic fingerprints (FP) and (FIG. 10B) using both the atomic fingerprints and charge density descriptors (FP+CHG).

FIG. 11A: Histogram of MAE from the five-fold cross validation for the potential energy prediction with three different input descriptors: 1) the atomic fingerprints (FP); 2) the charge density descriptors (CHG); 3) the atomic fingerprints along with the charge density descriptors (FP+CHG). Plots of the two main principal components (PC) from only using (FIG. 11B) the atomic fingerprints and using (FIG. 11C) both the atomic fingerprints and the charge density descriptors. The points are colored with respect to the total potential energy.

FIGS. 12A-12C: Comparison of ML-DFT and SIMPLE-NN. RMSE for the potential energy (FIG. 12A), atomic forces (FIG. 12B), and stress (FIG. 12C) predicted with trained ML-DFT and SIMPLE-NN following the same five-fold cross validation study, using the polymer chains for training and crystal structures for transferability to new structures. Black vertical lines indicate the standard deviation.

FIGS. 13A-13C: Potential energy absolute errors. Histograms of the absolute error between the reference DFT and the predicted ML-DFT potential energies per atom for the three different types of structures studied.

FIG. 14: Potential energy curves for butane molecule with respect to the C—C distance of the central C—C bond. Grey region indicates the C—C distance sampled within the training and test database for butane. Rectangular outlines indicate the specific configurations used to plot the charge density differences shown and ϵ_{ρ} (%).

FIG. 15: Potential energy curves for but-2-ene molecule with respect to the C—C distance of the central C═C double bond. Grey region indicates the C—C distance sampled within the training and test database for but-2-ene. Rectangular outlines indicate the specimen configurations used to plot the charge density differences shown and ϵ_{ρ} (%).

FIGS. 16A-16B: Energy conservation in ML-DFT. Difference between the sum of all total forces and the minimum value of the sum, for (FIG. 16A) butane and (FIG. 16B) but-2-ene, with respect to the C—C distance of the central C—C bond. Arrow indicates the location of the minimum in the potential energy with ML-DFT.

FIG. 17: Atomic charge (L1) and RMSE of atomic forces of the C atom (L2) in six different configurations of the methane molecule where the distance of the H atom on the left to the central C atom is reduced.

FIGS. 18A-18D: Performance of the energy, stress tensor components, and atomic forces from the ML-DFT model for the test configurations of the entire database of molecules, polymer chains, and polymer crystals according to an exemplary embodiment of the present invention. FIG. 18A: Parity plot of the potential energy per atom. FIG. 18B: Parity plot of the six different components of the stress tensor. FIG. 18C: Parity plot of the atomic forces for each type of element. FIG. 18D: Histogram of the error between the reference atomic forces (F; (DFT)) and the predicted atomic forces (F; (ML)).

FIGS. 19A-19I: Performance of the ML-DFT DOS model according to an exemplary embodiment of the present invention. FIGS. 19A-19F: DFT DOS and ML-DFT DOS for test configurations of six different molecules and polymer chains. The DFT and ML-DFT VBM/CBM predictions are included. The vertical dashed dark line indicates the vacuum energy used as the global energy reference. FIGS. 19G-19I: Parity plots of the ML-DFT VBM, CBM, and resulting E_{gap} for the test configurations.

FIG. 20: Atomic accuracy of the ML-DFT DOS model. Cumulative density of the ratio between the absolute error in the ML-DFT prediction DOS DFT deviation as the absolute difference between the DFT value and the average of the DFT DOS. The dashed line represents the value where the ratio is 1.

FIGS. 21A-21B: ML-DFT DOS derived properties. Parity plots of the total valence electrons obtained from the DOS (FIG. 21A) and of the kinetic energy per atom of the independent valence electrons obtained from the DOS (FIG. 21B). Vertical lines indicate the standard deviation due to the dropout layers employed in ML-DFT DOS model.

FIG. 22: Total CPU time of DFT versus ML-DFT for electronic structure predictions according to an exemplary embodiment of the present invention. DFT shows an initial high computational cost and a cubic dependence on the system size. ML-DFT is orders of magnitude faster than DFT and linearly dependent on the system size. Squares: structures with carbon and hydrogen. Star: structure with three elements. Cross: structure with all four elements.

FIG. 23 is an exemplary computing device according to an exemplary embodiment of the present invention.

### DETAILED DESCRIPTION OF THE INVENTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

It should also be noted that, as used in the specification and the appended claims, the singular forms “a,” “an,” and “the” include plural references unless the context clearly dictates otherwise. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” may be used herein to connote different aspects of methods employed, the term should not be interpreted as implying any particular order among or between various steps herein disclosed unless and except when the order of individual steps is explicitly required.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter. Additionally, the components described herein may apply to any other component within the disclosure. Merely discussing a feature or component in relation to one embodiment does not preclude the feature or component from being used or associated with another embodiment.

In embodiments disclosed herein, the KS equation can be handled in a novel manner using a deep learning scheme, both in terms of methodological advancements and applicability, which can predict the electron density first and then can employ it as an additional descriptor of the material to further predict other electronic and atomic quantities. The electronic quantities predicted other than the charge density can be the density of states, valence band maximum (VBM), conduction band minimum (CBM), and band gap (E_{gap}). The atomic or global quantities predicted can be the total potential energy, atomic forces, and stress tensor, essential for molecular dynamics (MD) simulations. The deep learning scheme can expand the flexibility and transferability of the model, allowing for training on molecules, 2D, and 3D systems within a large chemical space composed of carbon (C), hydrogen (H), nitrogen (N), and oxygen (O). Overall, an exemplary embodiment of the present invention allows for a near-complete DFT emulation within a practical context, surpassing previous works in terms of methodological advancements as well as expanding the portfolio of predicted quantities.

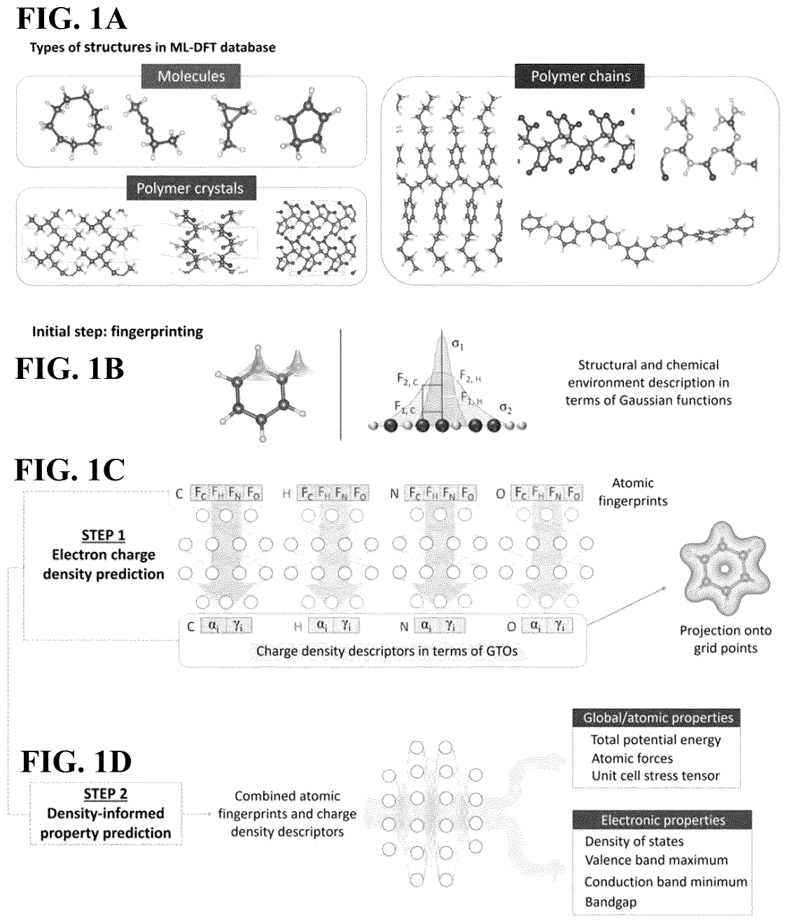

FIGS. 1A-1D show several components which are part of a machine learning (ML) workflow disclosed herein. The ML workflow can be described as a sequence of steps involving an ML system. The ML system, in some embodiments, can include a neural network or a deep neural network. In some embodiments, the ML system can include one or more neural networks. A reference, or “training”, data set, composed of molecules, polymer chains, and polymer crystal structures containing C, H, N, and O, and their corresponding properties can be computed using traditional DFT, and can be provided to the ML system. In some embodiments, the training data set can be indicative of DFT data for a plurality of materials. In an exemplary embodiment, the plurality of materials can include any combination of molecules, polymer chains, and polymer crystal structures comprising C, H, N, and O. In some embodiments, the DFT data can represent atomic configurations for the plurality of materials. Each reference atomic configuration can be represented by fingerprints using a ML-friendly atomic fingerprinting scheme. In some embodiments, the fingerprints can be determined for the atomic configuration for each of the plurality of materials. In an exemplary embodiment, atom-centered AGNI fingerprints can be used, which can represent the structural and atomic-level chemical environment of each atom in a machine-readable form such that it can be translation, permutation, and rotation invariant. Because not all properties predicted may be rotation invariant, i.e., electron density, atomic forces, and stress tensor, an intermediate internal reference system can be defined that can allow any quantity to be transformed to its value in the reference system of choice. To establish a direct (and nonlinear) mapping of the fingerprints (the “input”) to the spectrum of properties mentioned earlier (the “output”), deep neural networks can be used. In some embodiments, the fingerprints can be inputted into the ML system.

In an exemplary embodiment, the present invention can be inspired by DFT, and particular prominence can be given to the electronic charge density. A two-step learning procedure can be followed. A first learning problem (first step) can involve predicting the electronic charge density given the atomic configuration. In some embodiments, the ML system can generate electron charge density data for the plurality of materials. The first learning problem can employ GTOs as descriptors of the electronic charge density, but a predefined basis set may not be used; the model can learn the most optimal basis from the data examples, thus expanding the flexibility of the model. Once the electronic charge density descriptors have been predicted, they can be supplied as an auxiliary input (along with the atomic configuration fingerprints) to predict all other properties (listed as the second step of FIG. 1C). This strategy is consistent with the core concept underlying DFT (that the electronic charge density determines all properties of the system). Furthermore, in practice, this two-step route also can lead to more accurate and transferable results. In some embodiments, the electron charge density data and the fingerprint data can be inputted into the ML system. The ML system can then, in some embodiments, generate one or more DFT properties of the plurality of materials.

### Results

### Database

An example of an exemplary embodiment of the present invention is disclosed herein. Organic materials composed of four atoms: C, H, N, and O can be focused on. A database containing 67 molecules, 178 polymer chains, and 55 polymer crystals composed of C—C single, double, and triple bonds, as well as aromatic rings can be created. To provide the ML system with sufficient examples of configurational diversity, random snapshots of each type of structure from DFT-based MD runs at high temperatures can be procured. For the molecules and polymer chains, the MD runs can be performed at 300 K, and for the polymer crystals the MD runs can involve temperatures from 100 K to 2500 K. Over 118000 structures for the training and testing of ML-DFT can be used. For each type of structure, the selected configurations can be divided into training and test sets, following a 90:10 split. The models can be trained using an 80:20 split of the training set between training and validation. All performance results can be computed using the independent test set.

For each structure type, the elements contained in the structures, the temperature at which the MD simulations were performed, and the total number of snapshots (configurations) used from the MD for training and testing can be indicated.

TABLE 1

| Structure Type | Elements | Temp. of MD | Snapshots |
|---|---|---|---|
| Molecules | C, H | 300K | 13400 |
| Polymer Chains | C, H | 300K | 16800 |
| | C, H, O | 300K | 2500 |
| | C, H, N, O | 300K | 11000 |
| Polymer | C, H | 100-2500K | 16800 |
| Crystals | C, H, O | 100-2500K | 36000 |
| | C, H, N, O | 100-2500K | 22050 |

Due to different restrictions within the various models in ML-DFT, the size of the training database in each case can vary:

Charge density model: Due to the complexity of the model, only those systems with 50 atoms or fewer per element can be employed during training. The larger systems can be used as transferability check. Therefore, the amount of structures employed in the training of the charge density model can be around 32000.

Potential energy, atomic forces, and stress components model: In this particular case, the entire database can be employed, reaching over 118000 structures.

DOS/VBM/CBM model: In this model, the vacuum containing structures can be used exclusively, eliminating the crystal polymers. Therefore, a database of around 41000 structures can be used.

All the DFT reference data calculations can be performed using the Vienna Ab Initio Simulation Package (VASP).

### Fingerprinting

In a continued example embodiment of the present invention, two different types of fingerprints or descriptors can be employed: the atomic (or structural) fingerprints and the electron charge density descriptors. The atomic descriptors can be the AGNI atomic fingerprints, which can describe the structural and chemical environment of each atomic configuration. The AGNI atomic fingerprints can be previously developed, and can have been used to create ML potentials and force fields for a variety of materials as well as in previous work on predicting the grid-based electronic structure and the atom-based density of states. The atomic fingerprints, computed for each atom, can combine scalar, vector, and tensor-like expressions by summing over various Gaussian functions, resulting in translation, permutation, and rotation invariant descriptors.

The predicted electronic charge density descriptors can constitute the second type of fingerprints. Once all the configurations have been fingerprinted, the AGNI atomic fingerprints can be used as input for the charge density model, which can predict the decomposition of the atomic charge density in terms of GTO basis functions. The set of optimal GTO basis functions can be selected by the model in terms of the exponent of the Gaussian along with the constant multiplying the GTO; initial decomposition of the reference charge density in terms of GTOs may not be performed. The error made during the training of the model can be computed by projecting each set of atomic GTO basis functions onto the same grid points used for the reference DFT charge density.

As the input atomic fingerprints can be translation, permutation, and rotation invariant, the predicted constants and exponents of the GTO basis functions decomposition can be within the internal reference system of the atom. Because the reference electron charge density may not be rotation invariant, a transformation from each atom's internal reference system to the global reference system of the electron charge density (the Cartesian system) can be required, before projecting the predicted charge density onto the grid points. The vectors for the transformation matrix of each atom can be defined using the two nearest neighbors (independent of the element type): first vector can be the one pointing to the first nearest neighbor, the second vector can be defined as perpendicular to the plane containing the central atom and its two nearest neighbors, and the third vector can be perpendicular to the first two vectors. All vectors can be normalized to obtain an orthonormal reference system.

### Internal Reference System for Each Atom

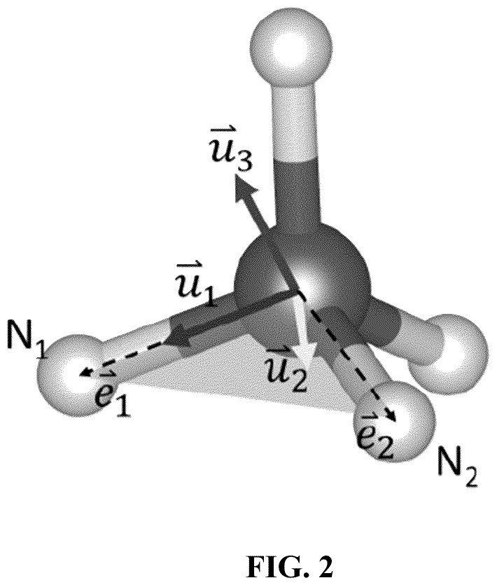

In a continued example embodiment, the internal reference system of each atom can be defined using the two nearest neighbors. FIG. 2 shows the methodology followed in defining the internal reference system for the carbon atom. The first and second nearest neighbors can be initially considered, identified by N₁ and N₂, respectively. The vectors can be:

$${e}_{1} = {r}_{N 1} - R \qquad Equation 1$$

$${e}_{2} = {r}_{N 2} - R \qquad Equation 2$$

with R the position vector of the carbon atom, and r_{N1} and r_{N2} the position vectors of the first and second nearest neighbors, respectively. Using e₁ and e₂, a third vector can be defined perpendicular to both of them as,

$${e}_{3} = {e}_{1} \times {e}_{2} \qquad Equation 3$$

Both e₁ and e₃ can be normalized, resulting in u₁ and u₃. The third vector to determine the orthonormal basis set can be defined as,

$${u}_{2} = {u}_{3} \times {u}_{1} \qquad Equation 4$$

The internal reference system of the carbon atom can then defined as I={u₁, u₂, u₃} which create the transformation matrix, I.

The resulting transformation matrix can be used to convert from the orthonormal internal reference system of each atom onto the Cartesian reference system, allowing the transformation of any rotation invariant value of a property (such as the decomposition onto GTOs) onto the Cartesian reference system. Because of the computational cost of projecting the GTOs onto grid points, the training can be restricted to structures with up to 50 atoms per element. Once the model is trained, the predicted constants and exponents for each atomic fingerprint can be referred to as charge density descriptors. Unlike the atomic fingerprints, which can be determined by a set of predefined equations, these charge density descriptors can be learned by the neural network and provide an electronic description of the system.

### Charge Density Decomposition and Neural Network Details

In a continued example embodiment of the present invention, to decompose the charge density into atom-centered contributions two different types of basis functions can be initially tested: Slater-type orbitals (STO) and Gaussian-type orbitals (GTO). This choice can be attributed to the effectiveness of the resolution of identity in describing the charge density. However, the present approach significantly differs from previous attempts at describing the electron density in three ways: (1) Although STOs and GTOs can be used, the neural network model of the example embodiment can learn the best basis functions for each element and atom depending on its atomic surrounding; in other words, the neural network can learn the constants multiplying each orbital as well as the exponents describing the radial part of the orbitals. Therefore, the example embodiment of the present invention may not necessarily be constrained by a predefined set of fixed basis functions in describing the charge density for a given element. (2) As an already available basis set of GTOs or STOs may not be required, the present approach may not require an initial charge density decomposition onto a predefined basis set and therefore, can learn the best decomposition by directly comparing to the ground state charge density from DFT, not by comparing with an already decomposed charge density carrying a decomposition error. (3) This much simpler, powerful, and accurate approach can apply for isolated molecules as well as condensed-phase or periodic systems.

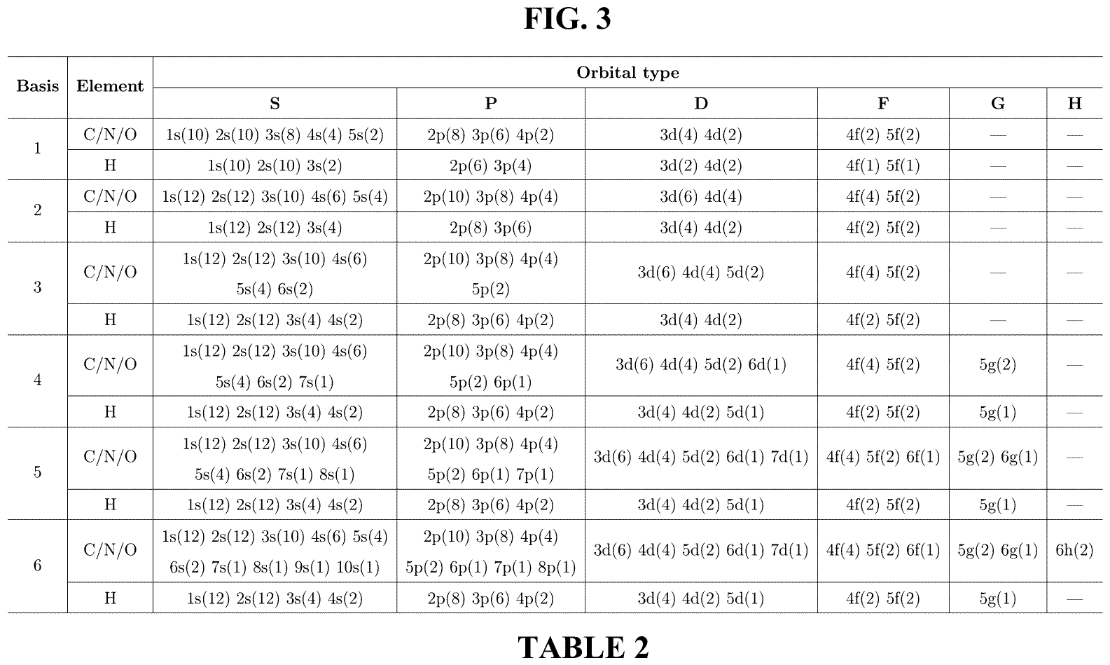

TABLE 2 is shown in FIG. 3 and illustrates the orbitals which can be used for each element within each basis set, with the number of functions (either GTOs or STOs) used to describe each in parenthesis.

An exemplary implementation of the protocol in the neural network scheme is as follows. First, the fingerprints of each atom are given as input into a 200-neuron four-dense layer neural network model. All dense layers have hyperbolic tangent activation function. The last output layer has a linear activation function. Each element type has different neural network with identical architecture. In this specific case, four different sub-neural networks are trained, one for each element. Within each sub-neural network, the fingerprints of each atom are mapped into the constants and exponents of the basis set functions. Regarding this point it is important to note that the user only needs to specify how many orbitals of each type (e.g. 1S, 2S, 3S, 2P, etc.) are desired.

In the approach, the comparison with the reference charge density can take place on the grid points. Due to the use of STOs and GTOs, a very small set of grid points from the millions describing each atomic configuration's charge density can be necessary. 1000 grid points from each atomic configuration can be used. These 1000 points can be randomly chosen from the total ordered by the charge density value, to ensure the charge density is uniformly sampled, focusing on the most important regions. During training, each atom's charge density contribution to the selected grid points can be calculated by projecting the predicted basis functions onto the grid points coordinates transformed to the internal reference system of the atom. Afterwards, all atoms' contributions can be summed and compared to the reference DFT charge density in the selected grid points.

However, due to the rotation invariance of the input atomic fingerprints, a transformation of the coordinates of the grid points can be required to ensure correct behavior. This can be done by using the internal reference system of each atom through the transformation matrix, I. Before projecting the predicted basis functions of each atom onto the grid points, the coordinates of the grid points in the global Cartesian reference system can be shifted to be centered around the atom and then transformed to the internal reference system as,

$${r}_{int} = I ( {r}_{g l o b a l} - R ) \qquad Equation 5$$

where rᵢₙₜ is the position vector of the grid point with respect to the internal reference system of the atom, I is the transformation matrix, r_{global} is the position vector of the grid point in the Cartesian reference system, and R is the position vector of the atom in the Cartesian reference system.

The 1000 grid points for the training of the charge density can be selected in the following manner: from the first 0.5% of the grid points with the highest charge density value, 50 are randomly selected; from the grid points within the 0.5% and 1%, 50 are randomly selected; between 1%-5%, 150 grid points; between 5%-10%, 200 grid points; between 10%-20%, 150 grid points; between 20%-30%, 100 grid points; between 30%-40%, 100 grid points; between 40%-50%, 100 grid points; between 50%-70%, 50 grid points; and between 70%-90%, 50 grid points. Randomly choosing the grid points in this manner can ensure that regions of high interest are sampled (e.g. those with high charge density) more densely, while the specific chosen grid points can vary from one atomic configuration to another. The higher the number of grid points chosen, the better (but also the more expensive) the training can be. Some tests with 200, 800, 1000, and 1600 grid points showed that 1000 are the best compromise between accuracy and computational expense. During testing, all grid points are used to compare with the DFT reference data.

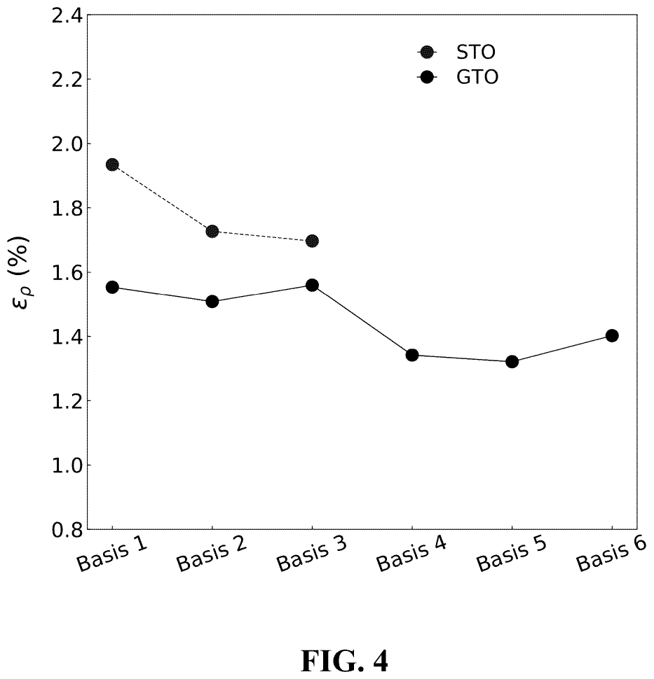

To decide which type of orbitals (STO or GTO) and which basis set is the most optimal, an initial test using the hydrocarbon polymer chains dataset can be performed. FIG. 4 shows the performance for each test. Given the error and the computational cost as the basis sets are increased, GTOs with Basis set 4 can be used.

### Charge Density Prediction

In a continued example embodiment of the present invention, to study the performance of the charge density model, the mean absolute percentage error (Ep) for each configuration can be computed as,

$${ϵ}_{ρ} ( \% ) = 100 \frac{{\sum }_{{ρ}_{M L} ( {r}_{j} )} \vert {ρ}_{D F T} ( {r}_{j} ) - {ρ}_{M L} ( {r}_{j} ) \vert}{{\sum }_{j} {ρ}_{D F T} ( {r}_{j} )} \qquad Equation 6$$

where ρ_{DFT}(rⱼ) and ρ_{ML}(rⱼ) are, respectively, the reference DFT charge density and ML-DFT charge density at grid point j, for the same configuration.

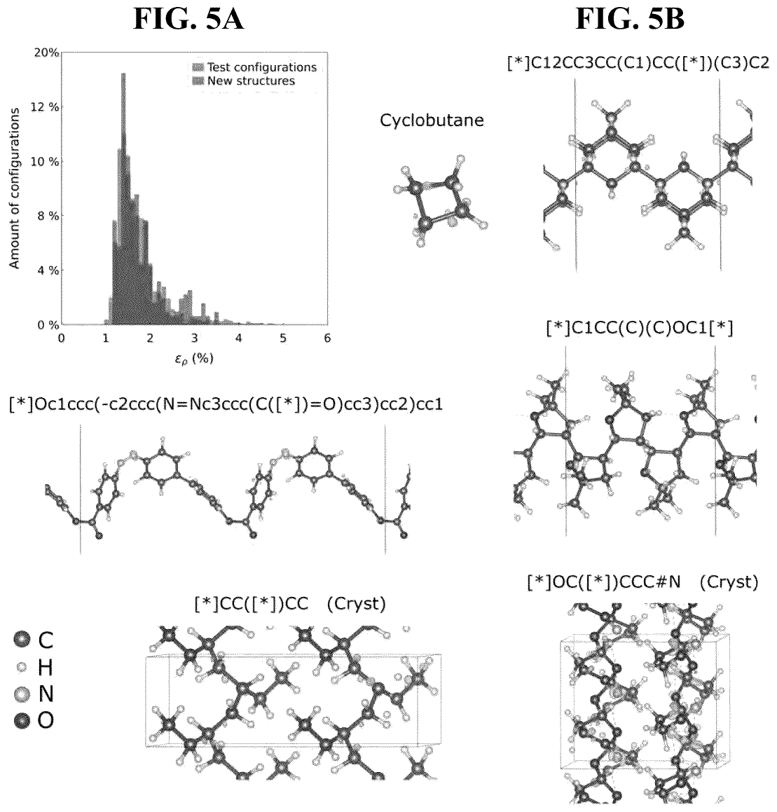

The accuracy of the charge density model can be observed in FIG. 5A, where ϵ_{ρ} for the test configurations of the training set ranges mainly from 1.0% to 3.0%, with a few cases extending up to 5.0%. Most notably, the performance on the new structures (more than 50 atoms per element) not included during training, can be very similar to those structures used for training. An overall performance value can be calculated as the mean of ϵ_{ρ} computed by summing ϵ_{ρ} for all configurations and normalizing by the number of electrons in each configuration. For the test configurations, the mean of ϵ_{ρ} is 1.75%, increasing slightly to 1.97% for the new structures.

FIG. 5B presents the valence charge density difference between the reference DFT and predicted ML-DFT for various atomic structures: the cyclobutane molecule, three different polymer chains, and two polymer crystals. The isosurfaces can occupy a very small volume, due to the high accuracy of the predicted ML-DFT charge density.

Various other charge-density dependent properties can be calculated from the predicted charge density and charge density descriptors, including but not limited to the partial atomic charges, the reduced density gradient for the analysis of non-covalent interactions, and the dipole moment. In addition, to further extend the applicability of the method to situations requiring the full electron density, ML-DFT can also provide the core electron density by mapping it to the DFT reference using Is orbitals, with an accuracy of around ϵ̅_{ρ,core} (%)=5×10⁻⁵. When the full electron density is used, the error of the predicted total electron density can be reduced by a 27% overall, with ϵ̅_{ρ,full}=1.28% on test configurations and ϵ̅_{ρ,full}=1.44% on new structures.

Properties Derived from Predicted Valence Charge Density
Partial Atomic Charges from Charge Density Descriptors

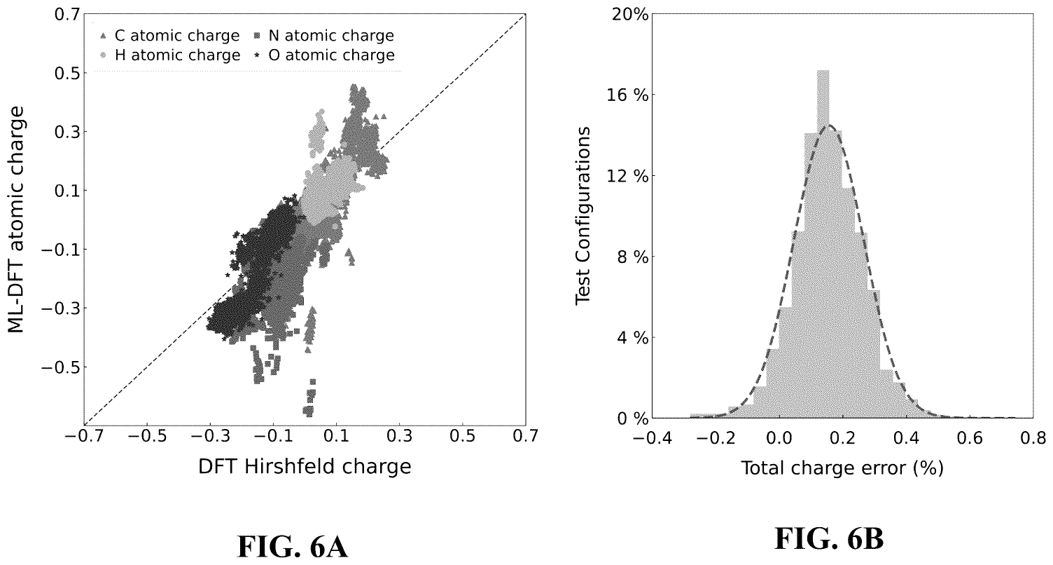

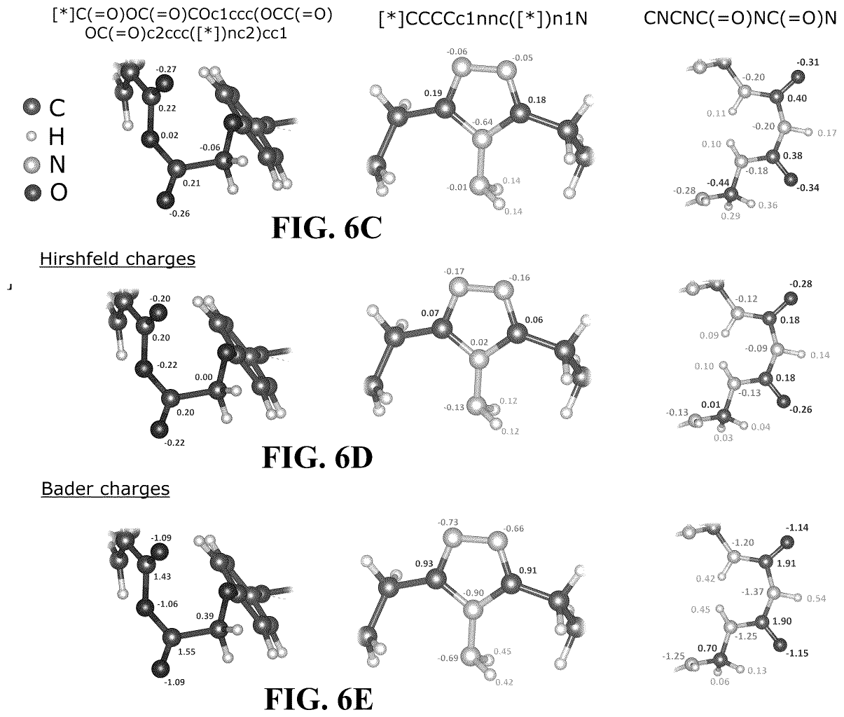

In an example embodiment of the present invention, the atomic charge of each atom can be easily calculated from the predicted charge density descriptors, without the need to project onto the grid points. FIG. 6A shows the parity plot between the DFT Hirshfeld charges and the ML-DFT predicted atomic charges. The charge density model can correctly predict the atomic charges and can learn the correct electronegative nature of each element: while N and O atoms can be mainly negatively charged with some cases showing a positive charge, the H atoms can be always positively charged, and C atoms can show both positive and negative charges.

The predicted charge density descriptors can have a physical meaning, and may not be just numerical artifacts. Additionally, in FIG. 6B shows the histogram of the total charge error between the DFT total charge and the ML-DFT total charge, as a percentage with respect to the total DFT charge. The results can vary between-0.3% and 0.7%, showing that the summation of the predicted partial charges can be very close to the correct total charge of the system, a condition which may not be impose during training.

It is important to note that, although the ML-DFT atomic charges can deviate from the Hirshfeld charges in some cases, it does not mean they are “incorrect”. There are multiple methods (each one non-unique) to calculate the atomic charges, and the results from some methods can differ a lot from others. Nevertheless, it has been reported that the Hirshfeld method provide values for the atomic charges that are more consistent with intuition than those obtained from Bader charge analysis, Mulliken or Lowdin charges.

To provide some quantitative evaluation to the difference between the ML-DFT and the DFT Hirshfeld atomic charges, FIGS. 6C-6E display three different cases where the ML-DFT atomic charges show the largest disparity with the DFT Hirshfeld charges, along with the calculated Bader charges from the DFT charge density. Even in those cases with the largest differences between ML-DFT and DFT Hirshfeld atomic charges, those differences can be much lower in value than the differences between DFT Hirshfeld and DFT Bader atomic charges. Therefore, the predicted charge density descriptors can provide a rapid means of calculating partial atomic charges while still maintaining a relative high accuracy when compared to other methods in the field.

### Reduced Density Gradient

In a continued example embodiment of the present invention, to further study the accuracy of the predictions the RDG calculated from both the DFT electron density and the predicted electron density can be compared. The RDG can be defined as

$$R D G = \frac{1}{2 {( 3 {π}^{2} )}^{1 / 3}} \frac{\vert {Δ}_{ρ} \vert}{{ρ}^{4 / 3}} \qquad Equation 7$$

where ρ is the electron density. Low RDG values with high ρ can correspond to covalent interactions, whereas low RDG values with low ρ can map to non-covalent interactions. However, to evaluate the attractive or repulsive nature of these non-covalent interactions the sign of the second density Hessian eigenvalue (λ₂) can also be required. This value can distinguish bonding interactions or H-bonding (λ₂<0) from non-bonding interactions or steric repulsive interactions (λ₂>0). Additionally, the regions with very low density values (ρ<0.005 a.u.) can correspond to weaker dispersion interactions such as van der Waals interactions.

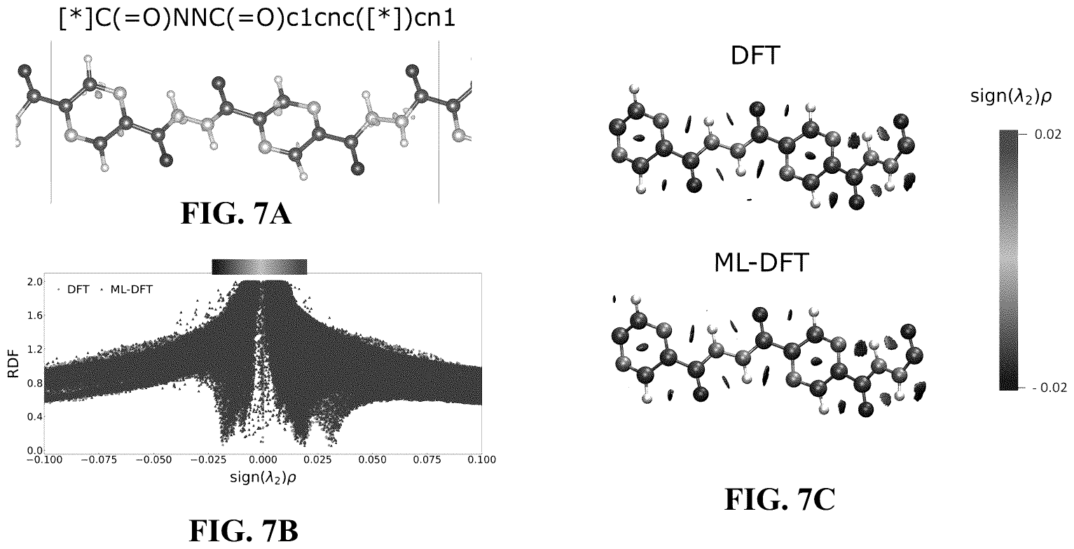

FIG. 7B shows a computed RDG diagram with respect to sign (λ₂)ρ using the DFT and ML-DFT charge densities, for the polymer chain in FIG. 7A. Noncovalent interactions (NCI) data can be computed using critic2 software, providing as input the charge density, either from DFT or from ML-DFT. The RDG values obtained from the predicted charge density can very closely follow those from DFT, with small deviations at low density. To visualize the differences due to these deviations, the NCI regions corresponding to RDG=0.5 can be plotted, colored by the value of sign (λ₂)ρ (FIG. 7C). The comparison between NCI plots can be exceptional, meaning that the ML charge density model does not necessarily only recover the correct value of the charge density with great accuracy, but also its derivative (see Equation 7).

### Dipole Moment Prediction

An example test using butane and propanol can be performed in an example embodiment of the present invention. By using propanol also it is possible to study the transferability of the trained charge density model to different systems not used during training (as molecular systems only hydrocarbon molecules may be employed), but with same type of chemistry (C, H, O, N). For butane, a test configuration from the database can be used and for propanol the relaxed molecule can be used, using the same DFT level of theory. The dipole moments can be computed in two different ways: using the charge density and using the atomic charges associated with each atom. TABLE 3 shows the errors in the predicted charge densities and the values of the dipole moment from DFT and from ML-DFT.

TABLE 3

| | | | Atomic Charges | | | Charge Density | | |
|---|---|---|---|---|---|---|---|---|
| | ϵ_{ρ} | ϵ_{ρ,n} | μ_{DFT} | μ_{ML-DFT} | μ_{ML-DFT,n} | μ_{DFT} | μ_{ML-DFT} | μ_{ML-DFT,n} |
| Butane | 1.219 | 1.233 | 0.048 | 0.046 | 0.046 | 0.570 | 1.480 | 0.444 |
| Propanol | 1.496 | 1.526 | 0.836 | 0.725 | 0.728 | 1.729 | 3.102 | 1.749 |

When using the predicted charge density to compute the dipole moment, the error with respect to the DFT value can be considerably higher than when using the predicted atomic charges. This can be justified, as during the training of the charge density model, the conservation of the total number of electrons in the system may not be imposed, and as a result, the sum of the predicted charge density may not be exactly the correct total number of electrons in the system (see FIG. 6B).

A potential solution is to multiply all values in the predicted charge density by a normalization factor defined as

$$n = \frac{Electrons ( DFT )}{E ; ectrpms ) ML - D F T )}$$

for each configuration. The normalized predicted charge density can vary slightly the atomic charges, but the modification can be very small.

The normalization factor for butane can be n_{butane}=1.00174 and for propanol nₚᵣₒₚₒₙₐₗ=1.00335. TABLE 3 includes the resulting dipole moments using the normalized predicted charge density and normalized atomic charges. As can be observed, this correction of the predicted charge density significantly can improve the dipole moment obtained from the charge density, while potentially having a minimal effect on the dipole moment from the atomic charges and on the error of the predicted charge density. Therefore, in future improvements of the ML-DFT model, this conservation of the total number of electrons during training of the charge density model may be imposed to ensure a correct calculation of the dipole moment from the predicted charge density, without the need of using a normalization factor.

### Core Electron Density Prediction

In the continued example embodiment, predicting the core electron density may not be an effort worth pursuing using complex machine-learning methods, as all non-all-electron-DFT can keep this frozen. It may only be necessary to learn the core electron density for an isolated atom of each element by fitting it to a set of Is orbitals. During the prediction of the total charge density of a given configuration within ML-DFT, the neural network model can be employed for the valence electron density and, for the core electron density, the projection of the same set of Is orbitals for each atom of a given element. Also, as s orbitals are spherically symmetric, there may be no need to employ the local reference system for the core electron density.

The complexity of learning core electron density can decay very fast with the distance to the center of the atom. Therefore, an extremely fine grid can be necessary in order to correctly capture the decay of the core electron density very close to the center of the atom, and the grid used for the valence electron density may not be fine enough.

In a continued example embodiment, for each element, DFT self-consistent calculations can be performed using an energy cutoff of 2500 eV for a single atom inside a box of 5 Å×5 Å×5 Å. Seven different configurations can be created, where the atom can be positioned at different locations to better sample the core electron density using the same grid. From the projected core electron density, 1000 grid points from each configuration can be selected, and the distance to the atom center (r) can be computed, as it may be the only value that is used in the s orbitals.

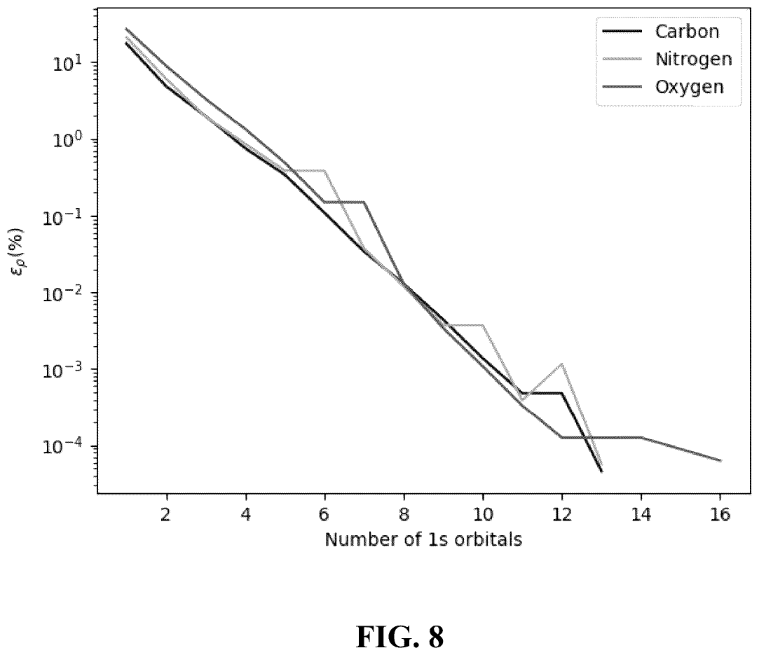

The grid points can be ordered with respect to the core electron density values and the highest 500 values can be selected, and then, from the 500 to 5000 ordered values 300 can be randomly chosen, and from the 5000 to the 50000 values, 200 can be randomly chosen. Using a simple curve-fitting approach, the r values can be fitted to the core electron density values using a varying number of 1s orbitals. In a similar manner as with the valence electron density and neural network, the curve-fitting approach can learn the optimal constants and exponents for the 1s orbitals. FIG. 8 shows the ϵ_{ρ} (%) depending on the number of Is orbitals used.

As can be observed in FIG. 8, the core electron density can be learned with extreme accuracy using only a few Is orbitals. In some embodiments, 13 1s orbitals can be used for C and N, and 16 for O, reaching accuracies of approximately ϵ̅_{ρ} (%)=5×10⁻⁵.

Using the fitted constants and exponents for each element, 25 different configurations from the dataset of molecules can be selected and polymer chains: 5 molecules, 5 hydrocarbon polymer chains, 5 polymer chains containing O, 5 polymer chains containing N, and 5 polymer chains containing O and N. For these 25 configurations, DFT self-consistent calculations can be run again including the core density, projected onto the same grid points as for the valence electron density in the work (using an energy cutoff of 500 eV). The mean Ep for the core electron density can be 0.0040%, significantly higher than obtained during fitting but still extremely small for practical use. When combining the error in the valence electron density and core electron density, the resulting ϵ̅_{ρ} can be reduced by a 27% (±2) with respect to the error obtained using only the valence electron density. Therefore, if applied to all the test configurations in the database, an error on the prediction of the total electron density of ϵ̅_{ρ}=1.28% and on the transferability test on new structures of ϵ̅_{ρ}=1.44% is possible.

### Charge Density Prediction on Delocalized Electronic Structure

In a continued example embodiment, to further test the applicability to more delocalized charge densities, a test using data from a short MD simulation of liquid Li at 500 K can be performed. In the test of the example embodiment, VASP can be used with a PAW potential and the PBE functional, with a kinetic energy cutoff of 250 eV and Gaussian smearing of 0.2 eV. The system can be composed of 90 atoms which can be first disordered by simulating at 2500 K for 1500 time steps. The disordered system can run another 1500 time steps at 500 K. The time steps can be of 1 fs. From the last 1500 time steps, the first 300 can be discarded, due to thermalization, and from the remaining 1200 500 can be randomly selected to create the database. The database can be randomly separated into 10 test configurations and 490 configurations for training and validation, which can be randomly separated using a 80:20 split.

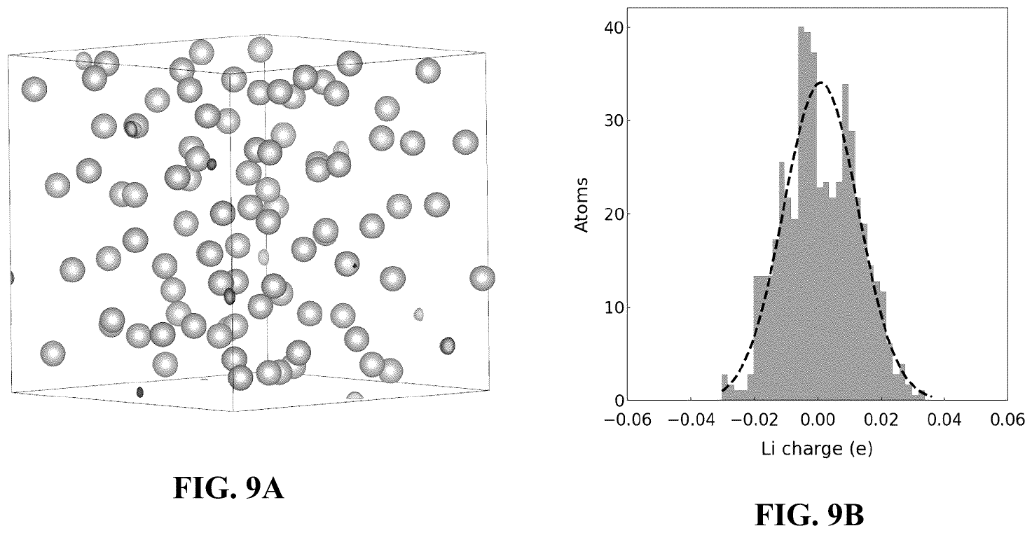

The same set of parameters for the atomic fingerprints can be used as in the manuscript, and can result in 90 atomic fingerprints for each Li atom. Using these fingerprints, the charge density model can be trained using the same type of GTO basis set as for H. Due to the small amount of training configurations, the architecture of the neural network can be reduced to the layers with 200 neurons each. The resulting charge density model can be tested on the ten test configurations obtaining ϵ_{ρ}=5.94%. FIG. 9A shows the valence charge density difference between DFT and ML-DFT for a test configuration. FIG. 9B depicts a histogram containing all the atomic charges of the Li atoms in all ten test configurations. As expected, in liquid Li, the mean value of all atomic charges can be 0. The histogram can be fitted by a Gaussian with a mean value of 0.00107 e.

### FP and CHG PCA Explained Variance

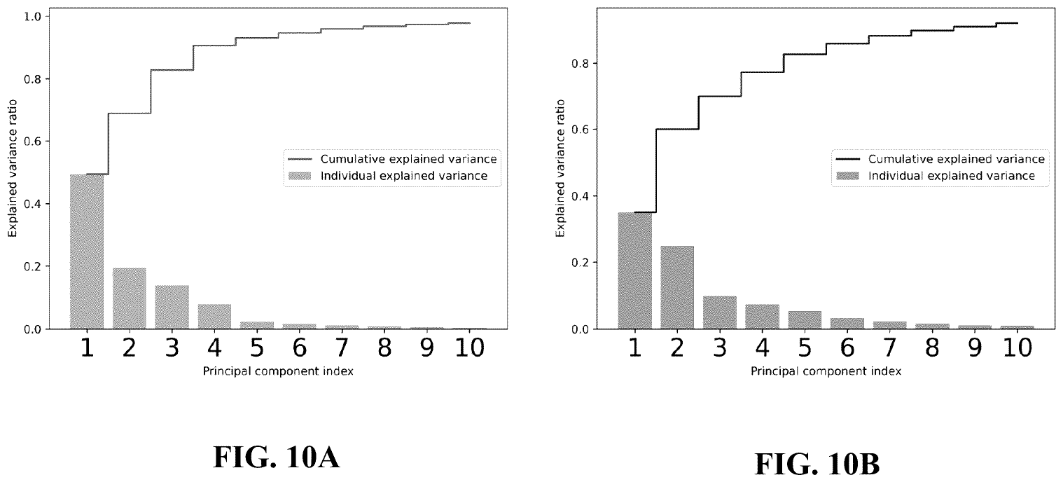

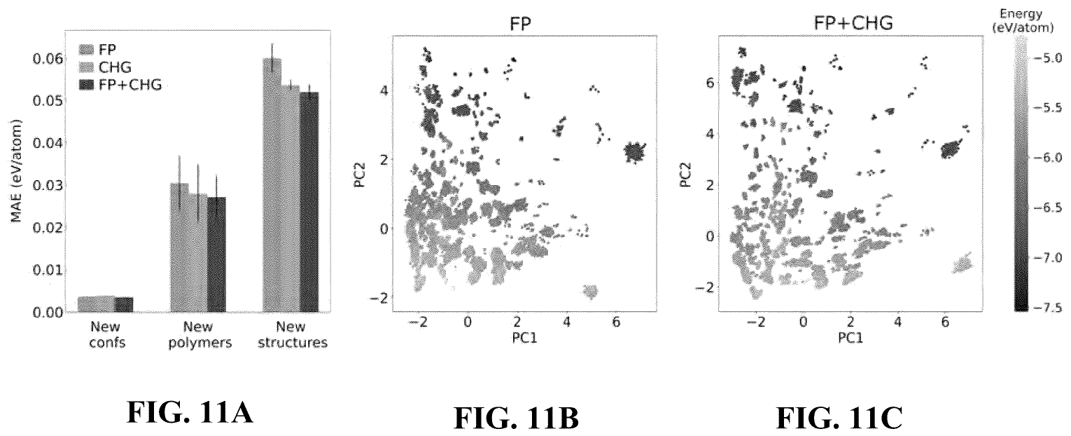

As observed in FIGS. 10A-10B, the cumulative explained variance of the first two PCA when using only the atomic fingerprints can be around 70%, whereas it may only reach 60% when using both the atomic fingerprints and charge density descriptors. This result, when combined with FIGS. 11A-11C, where the variance of the first two PCA can be larger in the case of using both the atomic fingerprints and charge density descriptors, clearly shows how the addition of the charge density descriptors allows for a better description and introduces more variance in the description of the atomic structure.

Important previous ML work to predict the charge density based atomic contributions required an initial decomposition onto predefined basis sets, introducing an additional error. The ML model was trained to predict the components of the decomposition for each atom, resulting in good predictions of the charge density for cases within the training space, but leading to a lack of transferability to new cases. The authors used the full charge density instead of only the chemically active valence charge density, which can be used in the work. This difference results in their work presenting lower errors in the charge density prediction, as the high-valued core charge density is easily predicted and effectively lowers the percentage errors. Moreover, the need for already available basis sets to decompose the charge density can become a hindrance to the applicability of this method to some elements. Comparison with more recent work predicting the valence electron density using an atom-centered approach, shows the methods disclosed herein have better accuracy. Another important comparison with recent work is the high error cases within the training space, which in this study extend up to a maximum of 5.02%, whereas in prior works reach up to 11%. Overall, the ML-DFT charge density surpasses previous methods in terms of accuracy and/or methodology, simplifying the protocol and, in the process, extending the applicability and transferability.

### Total Potential Energy, Atomic Forces, and Stress Tensor

In a continued example embodiment, DFT posits that the ground state charge density can have a one-to-one mapping with the ground state potential energy. Similarly, in the ML-DFT emulator, once the atomic charge density descriptors are predicted, they can be used as input (along with the AGNI atomic fingerprints) to predict the potential energy, atomic forces, and stress tensor. To evaluate the improvement on the accuracy and transferability of the potential energy model by including the charge density descriptors in the input layer, three different options can be considered: using the atomic fingerprints only, the charge density descriptors only, and the atomic fingerprints and charge density descriptors together. The polymer chains can be used only for training, and the polymer crystals can be left to test the transferability to new structures. A five-fold cross validation can be performed, and the performance can be evaluated on test configurations for the polymer chains used during training (a.k.a. new configurations), for new polymer chains (a.k.a. new polymers), as well as for polymer crystals (a.k.a. new structures).

FIG. 11A shows the histogram with the mean and standard deviation of the mean absolute error (MAE) value of the total potential energy per atom of each type of descriptor on the test configurations. The combined atomic fingerprints and charge density descriptors can improve the accuracy and transferability and can reduce the deviation of the predictions, resulting in more robust models. As can be seen in the Principal Component Analysis (PCA) plots in FIGS. 11B-11C, the addition of the charge density descriptors can result in a better separation of the structures with different potential energies, thus improving the prediction capabilities of the model.

In the methods disclosed herein, the potential energy, atomic forces, and stress tensor components can each be predicted directly (without using one to derive the others) by employing the same transformation matrix from the charge density model to transform the atomic forces and stress tensor components into the Cartesian reference system. This can allow for a significant reduction of errors in the atomic forces and stress tensor components, and can improve the transferability to new structures.

### Potential Energy, Forces, and Stress Tensor Neural Network Details

In a continued example embodiment, the predicted charge density decomposition of each atom onto the basis set functions can be considered as a new set of translation, permutation, and rotational invariant fingerprints describing the system. The new set of fingerprints can be concatenated to the initial atomic fingerprints to create a more complete description of the atomic environment of each atom, and can be used as input to the neural network predicting the total energy, atomic forces, and stress tensor components.

Similar to the charge density model, the atomic properties model can be composed of four different sub models, one for each type of element. Each sub model can be composed of five dense layers with 300 neurons each. All dense layers have the L2 regularizer with 0.1 and the hyperbolic tangent activation function. The output layer with 10 neurons and the linear activation function can correspond to each atom's contribution to the total potential energy, its three atomic force components, and the six independent stress tensor components. The atomic forces and stress tensor components can be expressed in terms of the internal reference system of the atom. Therefore, the transformation matrix of each atom can be employed to transform each component to the Cartesian reference system, taking into account the vectorial and tensor nature of each case as follows,

$${F}_{g l o b a l} = {I}^{T} {F}_{int} \qquad Equation 8$$

$${S}_{g l o b a l} = {I}^{T} {S}_{int} I \qquad Equation 9$$

where F_{global} and S_{global} are the atomic forces and stress tensor in the Cartesian reference system and Fᵢₙₜ and Sᵢₙₜ are the atomic forces and stress tensor in the internal reference system of the atom, as obtained from the output layer of the neural network. I is the transformation matrix.

Afterwards, for the potential energy, the contributions of all the atoms can be summed, normalized by the number of atoms in the configuration, and compared to the normalized reference DFT total energy. For the atomic forces, the comparison can be made directly to the DFT atomic forces. For each stress tensor component, the contributions from all atoms can be summed and normalized to the total number of atoms in each configuration. Due to the difference in order of magnitudes between the atomic properties, when computing the overall mean-squared error (MSE) during training, each term (total potential energy, C forces, H forces, N forces, O forces, stress tensor components) can be weighted so that they contribute similarly. Specifically, the MSE from the potential energy per atom (in eV) can be multiplied by 105, the MSE from the atomic forces (in eV Å⁻¹) of each element can be multiplied by 10, and the mean MSE from the six stress tensor components (in KB) can be multiplied by 0.1.

### ML-DFT Versus SIMPLE-NN: A Comparative Study

To quantitatively compare the improvement on the predicted potential energy, atomic forces, and stress tensor components using the model against another similar capability, the same five-fold cross validation study can be performed using the SIMPLE-NN package, which can derive the atomic forces and stress from the potential energies. SIMPLE-NN is an open-source package that can generate ML potentials based on neural networks using the Behler-Parrinello type symmetry functions as descriptors for the atomic and chemical environment. In some embodiments, 86 Behler-Parrinello descriptors can be used for each element, and optimized the model architecture to 5 layers of 100 neurons each, using also the hyperbolic tangent activation function. The weights for each MSE during training in SIMPLE-NN can be the same as for the training of the ML-DFT model.

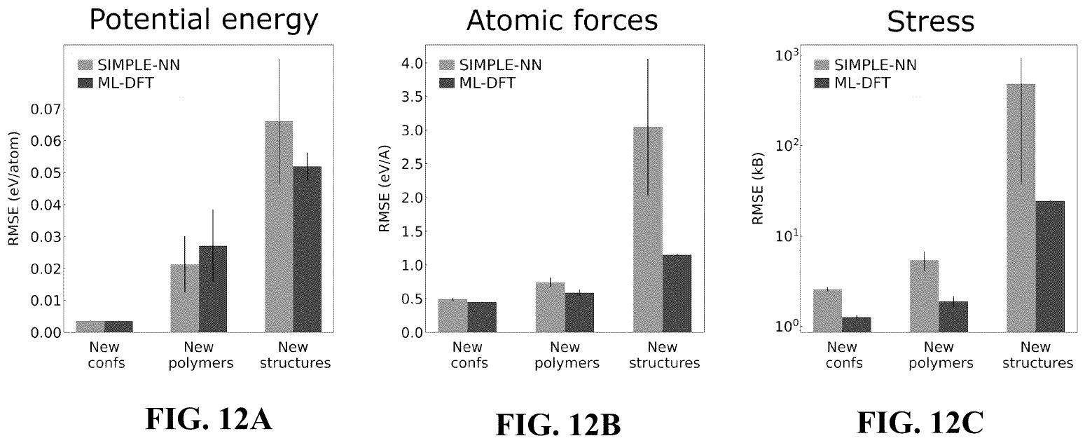

As can be seen in FIGS. 12A-12C, the RMSE of the total potential energy (FIG. 12A) for the test configurations within the same training space (‘New confs’), can be very similar for both the SIMPLE-NN and the ML-DFT models. When making predictions on new polymer chains (‘New polymers’), the RMSE of SIMPLE-NN can be lower than for ML-DFT. However, this is the only instance in which SIMPLE-NN outperforms ML-DFT. When making predictions outside the training space, for crystal polymers (‘New structures’), ML-DFT can show significantly lower RMSE, as well as much lower standard deviations. Analogous behavior can be observed for the atomic forces (FIG. 12B), where ML-DFT can outperform SIMPLE-NN in all cases, although the difference for the new configurations can be very small. The most outstanding results appear for the prediction of the stress (FIG. 12C). In this case, ML-DFT can significantly outperform SIMPLE-NN, by orders of magnitude (Y axis is in logarithmic scale) for the case of polymer crystals (‘New structures’).

### Potential Energy Absolute Errors

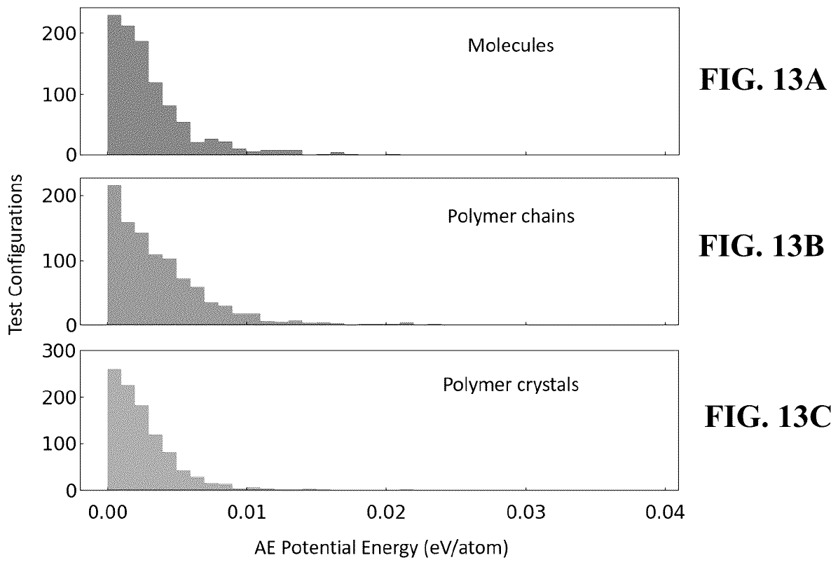

The three histograms plotted in FIGS. 13A-13C show the distribution of the absolute errors in the predicted potential energy. Although the polymer chains can present the largest errors, all results are within chemical accuracy.

### Bond-Breaking and Energy Conservation

Although the chosen database may not contain bond-breaking situations, it is interesting to study how the far from equilibrium can ML-DFT be trusted.

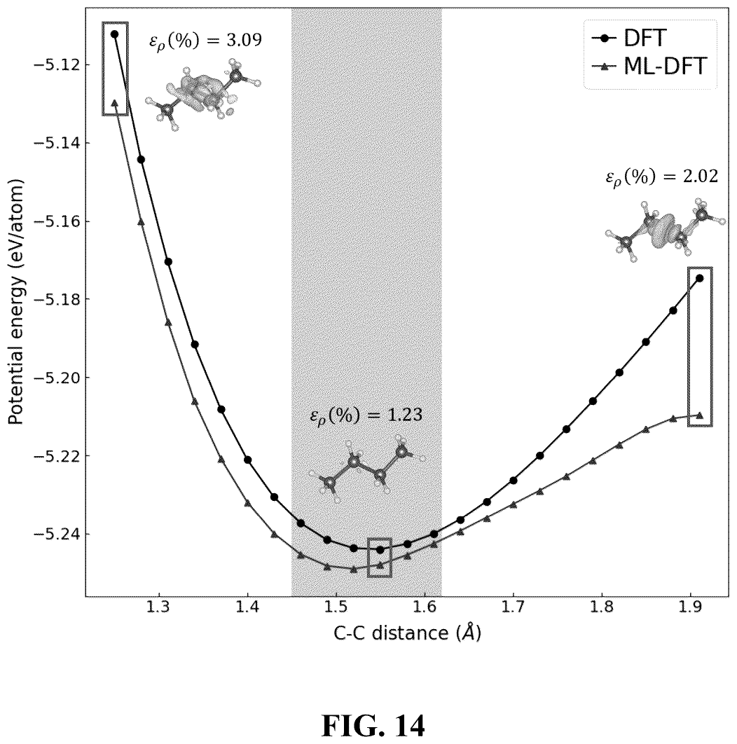

The limits of the model for butane and but-2-ene can be tested by predicting the total energy of the molecules as the distance of the central C—C bond is increased until broken, and also shortened. FIG. 14 represents the reference DFT and predicted ML-DFT energy curves with respect to the C—C distance of the central C—C bond for butane. Also included is the charge density difference between DFT and ML-DFT for three different distances shown with a rectangle in the energy curves, and the corresponding Ep (%).

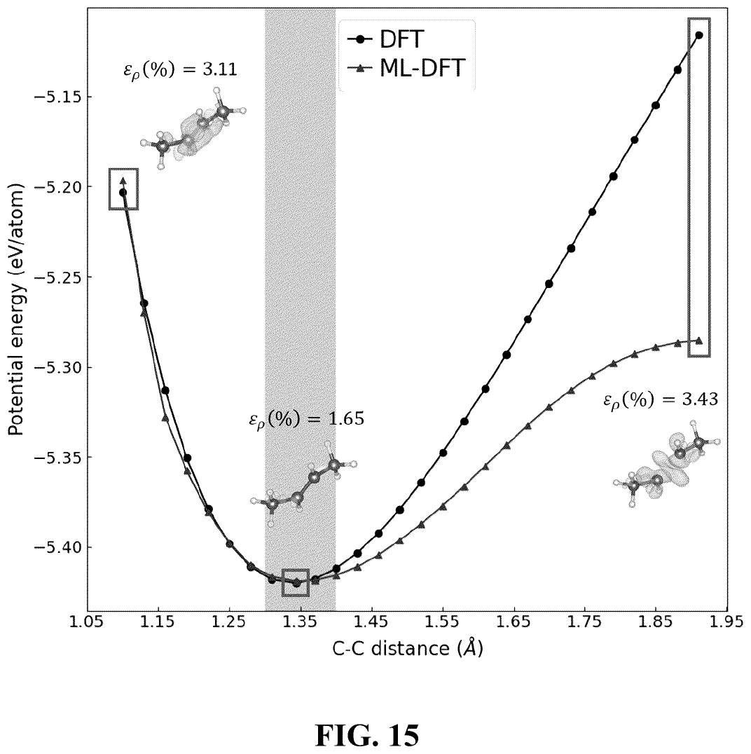

A similar test on but-2-ene can be performed to analyze the performance of the model far from equilibrium in the case of a double C—C bond. FIG. 15 represents the reference DFT and predicted ML-DFT energy curves with respect to the C—C distance of the central carbon double-bond for but-2-ene, along with the charge density difference between DFT and ML-DFT for three different states.

FIGS. 14-15 show that the ML-DFT model can perform better for situations where the bond is shortened, especially for potential energy calculations. For cases where the bond has a length longer than the cases sampled in the training database, the model's prediction of the potential energy can get worse. 2) The ML-DFT prediction of the charge density, although potentially worse for cases outside the training database, can still maintain an error that points to the robustness of the charge density model, even for significantly different situations such as the bond breaking of a simple and double C—C bond.

These two tests can be used to analyze the conservation of energy in the ML-DFT model, and if the predicted total forces of the system are minimum (not zero as it is not the fully optimized geometry) when the potential energy is also minimum. The total forces of all atoms in the molecules can be computed for a few configurations near the minimum of potential energy in FIGS. 14-15.

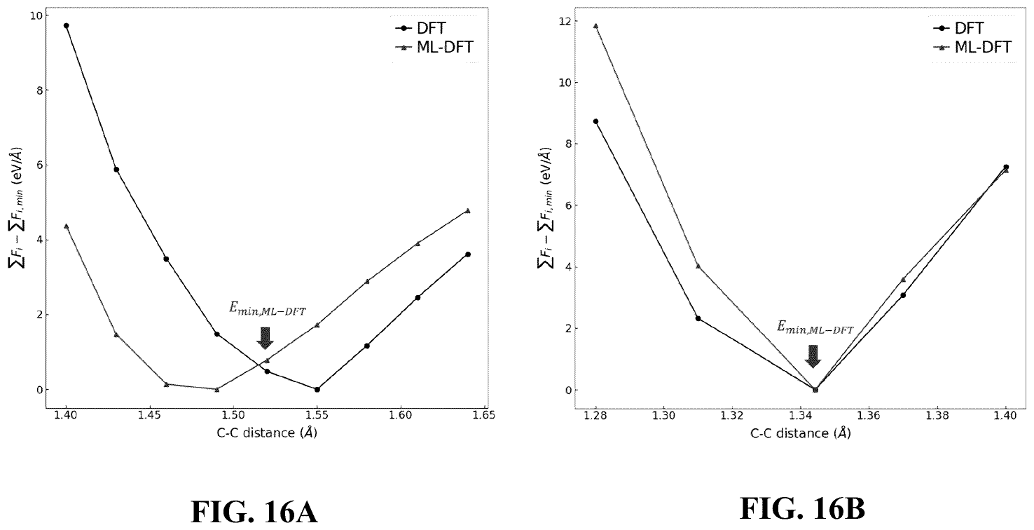

FIGS. 16A-16B depict the total forces obtained from DFT and ML-DFT for the same butane and but-2-ene molecules, respectively. To case the comparison between both DFT and ML-DFT results, each set of values has been shifted by subtracting the minimum value, ΣF_{i,min}. The location of the minimum potential energy predicted with ML-DFT is included.

In the case of but-2-ene, FIG. 16B, the minimum in the predicted atomic forces can fall on the same configuration as the minimum in the predicted potential energy, and very close to it in the case of butane, FIG. 16A. In addition, the curves followed by the predicted total forces can be very similar in both cases to the curve followed by the reference atomic forces. From FIGS. 16A-16B it can be concluded that by using the same neural network to predict the potential energy, atomic forces, and stress tensor, information regarding their relationship can be captured indirectly.

### Internal Reference System Stability: Atomic Forces and Atomic Charge

Although the definition of the internal reference system can give rise to a discontinuity when the order of the two closest neighbors is inverted, it may not be the cause for the error obtained in the predicted valence electron density nor in the atomic forces. Using a simple case, the methane molecule, a careful analysis can be performed on the carbon atomic forces of the effect of changing its internal reference system. This can be performed by slowly approaching the furthest H neighbor.

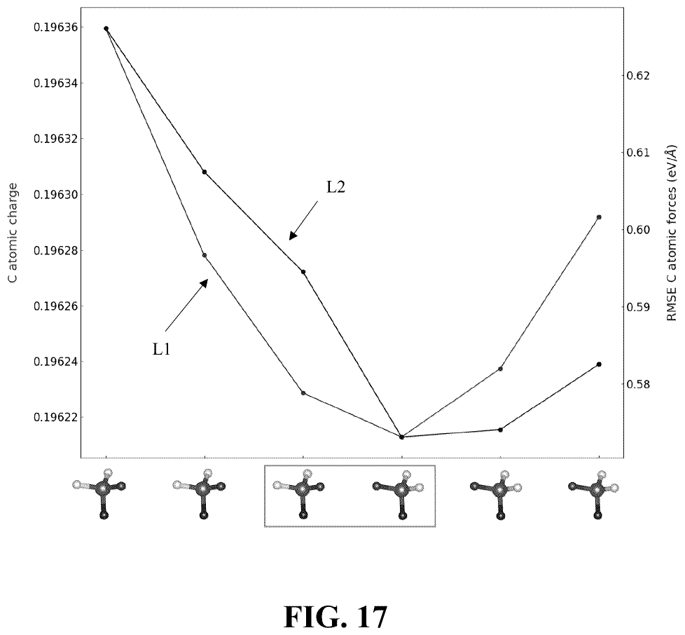

As illustrated in FIG. 17 the atomic charge of C (left Y axis) and the RMSE of the C atomic forces (right Y axis) can be represented with respect to six different configurations. A difference between the configurations can be the distance of the H neighbor (at the left of molecule), which can start being the furthest neighbor from C, and can slowly approach the C atom and can become the second neighbor, changing the internal reference system of C. As can be observed in FIG. 17, the change in the internal reference system can occur from snapshot 3 to snapshot 4 (enclosed in the rectangle). However, the RMSE in the C atomic forces may not present a drastic change with respect to the previous RMSEs.

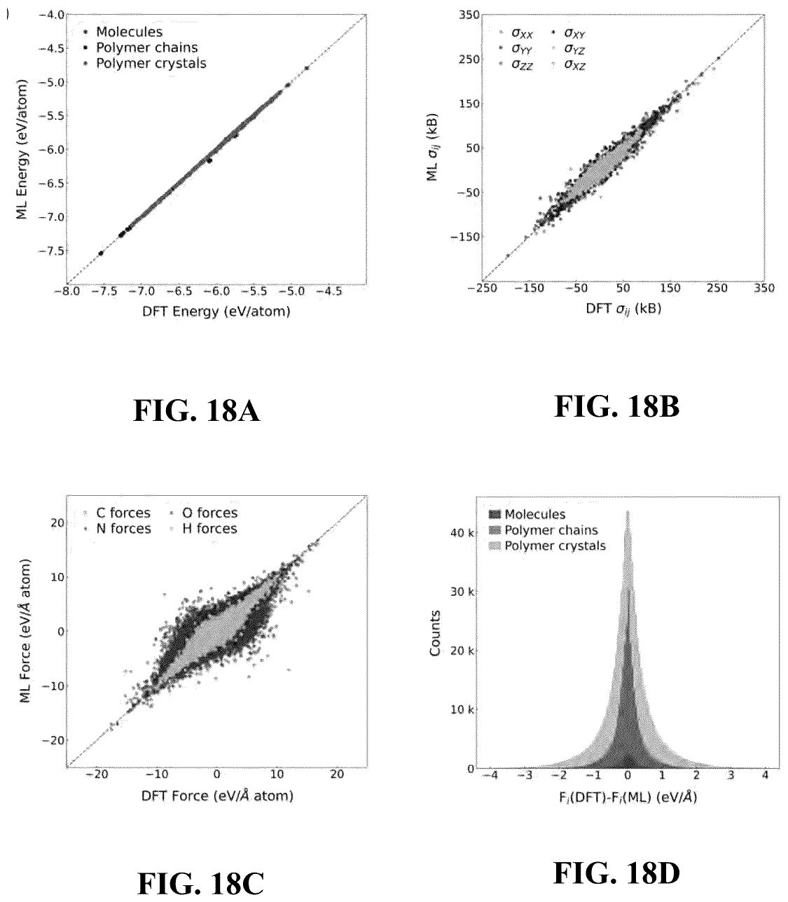

After confirming the advantage of employing the fingerprints and the charge density descriptors together, the energy, forces, and stress tensor model can be trained on the entire training set of molecules, chains, and crystals. FIGS. 18A-18D show the performance of the model on the test configurations for the atomic potential energy per atom (FIG. 18A), the stress tensor components (FIG. 18B), and the atomic forces (FIG. 18C). Both the potential energy and stress tensor components can be predicted with great accuracy, with a MAE of 3.3 me V/atom for the potential energy, and a mean root-mean-squared error (RMSE) of 6.182 kB for the diagonal stress components. However, the predicted atomic forces can present a mean RMSE of 0.759 eV/° A, with the C atomic forces presenting larger deviations from the reference DFT values, than the other elements studied. This deviation can be mainly observed in the polymer crystals, as observed in FIG. 18D, from the histogram of the error in the predicted forces most of the errors can be contained within ±1 eV/° A. There can be very few instances with errors larger than ±3 eV/° A, with the highest error obtained at ˜11 eV/° A.

Some possible reasons behind these high errors in the atomic forces could be attributed to the insufficient sampling of highly disordered structures present in the crystal polymers, as well as the inability to capture non-local effects, such as the long-range van der Waals dis-persion forces, using local atomic fingerprints. Similar results with large errors in the atomic forces have been reported in other previous studies on machine-learned atomic potentials and force fields for pure carbon structures and carbon-containing structures, where various methods of database optimization are used, such as active learning. These methods can also be used with ML-DFT to improve the performance in the atomic forces.

### Density of States Predictions

As previously mentioned, the solution of the KS equation in DFT describes the electronic structure of the system in the form of the charge density and the DOS. This last property can be essential for computing multiple electronic properties of the system, such as the VBM, CBM, and E_{gap}. Using a similar approach as previously described for the potential energy, the ML-DFT DOS predictor can also employ as input both atomic fingerprints and charge density descriptors. Following previous work from the inventors, the reference DOS curve can be previously shifted with respect to the reference energy of vacuum, and discretized every 0.1 eV from −33 eV to 1 eV. Due to this constraint, the model can be trained using vacuum-containing structures: molecules and polymer chains.

To achieve the highest accuracy in the VBM and CBM, and consequently the bandgap, their DFT reference values can be obtained directly from the (shifted) eigenvalues, and not from the smeared DOS. Due to the intensive nature of the VBM and CBM, the entire DOS/VBM-CBM model can first predict the smeared DOS curve as the sum of each atomic contribution, can normalize the total DOS with respect to the number of valence electrons, and can feed it to a second sub-neural network which can predict the VBM and CBM.

### Density of States and VBM/CBM. Descriptors and Neural Network Details

The combined atomic fingerprints and charge density descriptors can also be optimal features to predict other electronic structure related properties besides the total energy, atomic forces and stress tensor, such as the density of states. In an exemplary embodiment, the prediction of the DOS curve can be separated from the prediction of the valence band maximum (VBM) and conduction band minimum (CBM). The main reason for this decision is to obtain the best accuracy for both classes of properties. By using a very small Gaussian smearing, the VBM and CBM can be obtained accurately from the DOS curve, but predicting the entire curve can be difficult for the neural network, and may result in sub-par performance for all cases. This challenge has already been highlighted by others, where using a Gaussian smearing of 0.3 eV seems to be an optimal solution for the case of Silicon (Si) and their DOS protocol with Gaussian Process Regression.

In a continued example embodiment, even though the DOS of polymer chains presents a more challenging shape than Si, it can also be observed that by using a Gaussian smearing of 0.3 eV, the neural network can be able to predict the DOS with sufficient accuracy. The neural network architecture can be very similar to the previous DOS model for graphene-derived structures with four dense layers of 600 neurons each with dropout layers of rate 0.1 in between. The final layer can be a dense layer with 343 neurons followed by a 1D convolutional layer with three filters of size 3, from which the average value can be calculated, resulting in a 341-size vector.

All dense layers can have the L2 regularizer with 0.1 and the ReLU activation function. Each element can have its own sub-neural network with identical architecture. The ML-DFT VBM/CBM sub-neural network architecture can consist of two dense layers of 100 neurons each with L2 regularizer of 0.01 and the ReLU activation function.

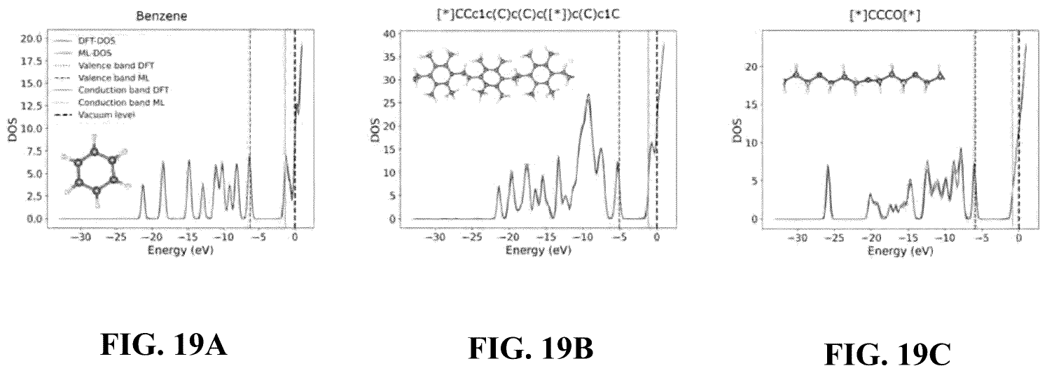

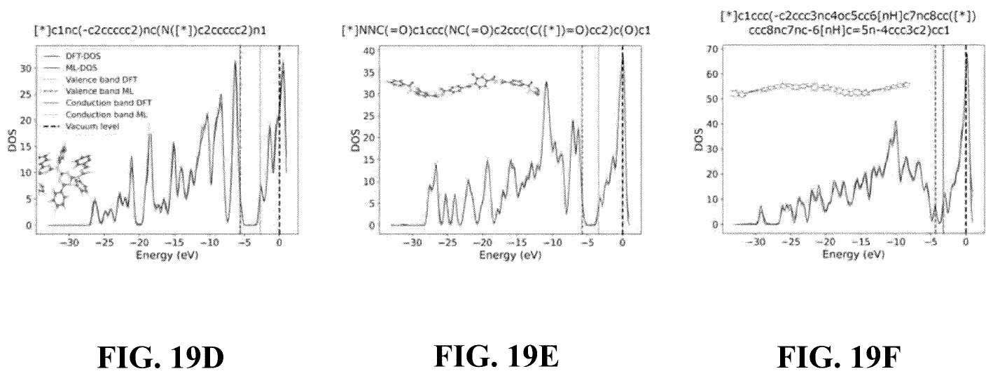

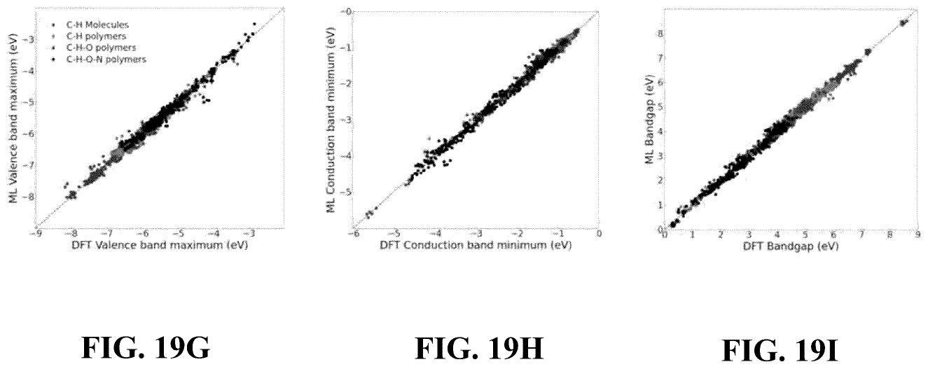

FIGS. 19A-19F display six different DOS for test configurations of different molecules and polymer chains. The test cases can possess a large variety of DOS curves. Nevertheless, the ML-DFT DOS model can accurately predict them. Also, the accuracy of the predicted DOS can be high enough to evaluate differences due to the atomic movement.

### Density of States Accuracy

To evaluate if the high accuracy obtained in the DOS prediction for the test configurations translates into a low enough error, another comparison can be performed. As all the training configurations can be taken from DFT-MD runs at 300 K, for each polymer, the mean DOS curve throughout the selected snapshots can be calculated. For each prediction, the ratio between the absolute error in the ML-DFT prediction and the absolute difference between the DFT value and the average of the DFT values for the snapshots can be computed; in other words, it can be assessed if the error made on the ML-DFT prediction is larger than the variance of the reference data due to atomic movements during DFT-MD.

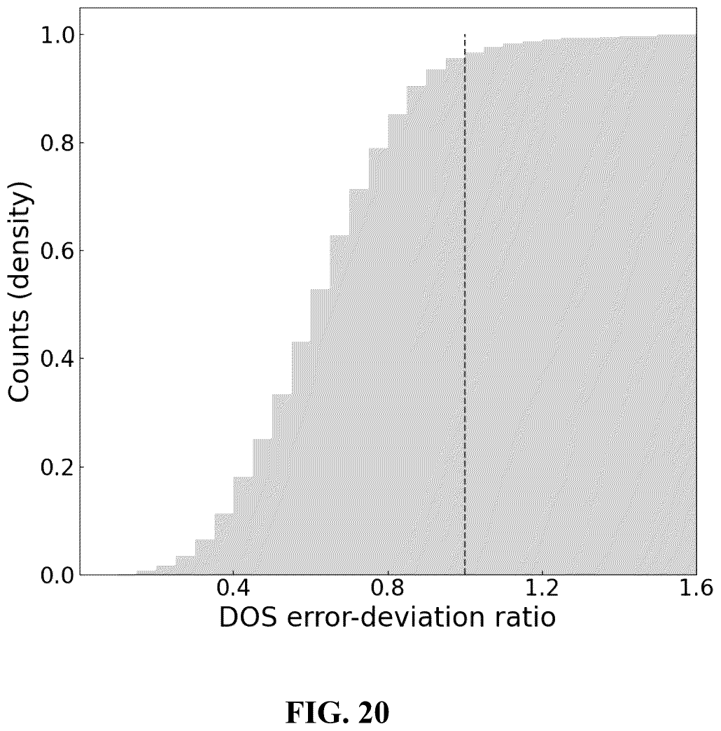

If the ratio is lower than one, then the ML-DFT DOS model can be accurate enough to predict changes in the properties due to small atomic movements. FIG. 20 shows the cumulative density of the error for all the test configuration and it can be seen that more than 90% of the configurations have a ratio lower than one (dashed line). Therefore, it may be concluded that the ML-DFT DOS model can be flexible and accurate even at the atomic scale.

### DOS Validation: Valence Electrons and Electronic Kinetic Energy

In a continued example embodiment, the condition on the integral over the occupied DOS may not be imposed during training, as opposed to previous work on using neural networks for carbon allotropes. The reasons for this decision are: 1) it may introduce spurious values in the DOS to compensate and obtain the correct number of electrons; and 2) the total potential energy can be directly obtained in the ML model and therefore, there is no need to use the predicted DOS to compute the kinetic energy of the non-interacting electrons. Therefore, obtaining the most accurate shape of the DOS for the molecules and polymer chains can be focused on, which can be a challenging task due to the wide variability in the reference data.

Nevertheless, both the integral over the occupied DOS and the sum over the KS energies can be computed, compared to the reference DFT energy, to check how these two properties are recovered by ML-DFT, even though the condition may not imposed.

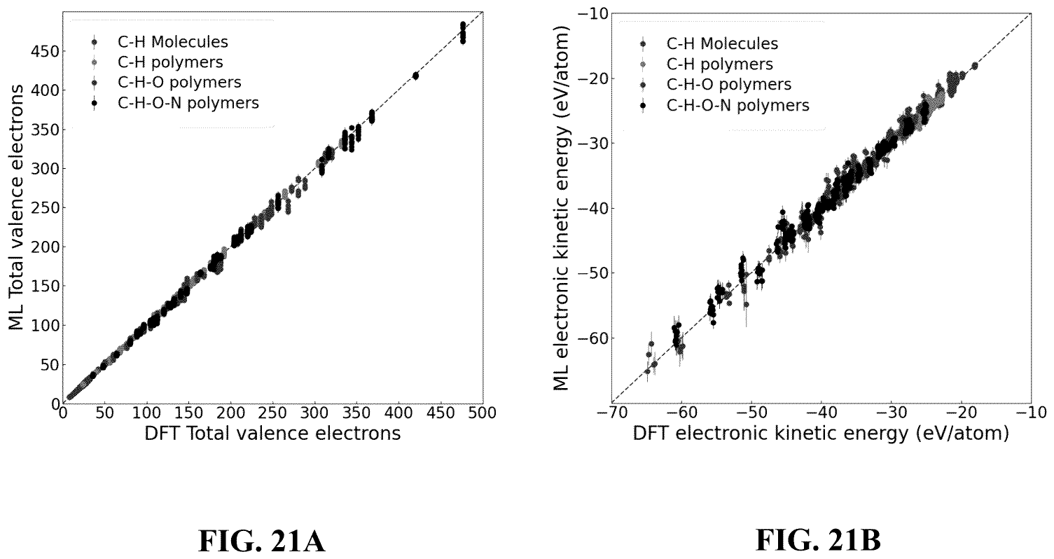

FIG. 21A shows the ML total valence electrons as the integral over the predicted DOS for all test configurations. In an exemplary embodiment, the ML-DFT model can learn the total valence electrons.

FIG. 21B shows the ML kinetic energy of independent valence electrons obtained from the predicted DOS, averaged over the total number of atoms in the system. In an exemplary embodiment, the comparison of the ML-DFT results with the DFT reference data can be reasonable, with a MAE of 0.523 eV atom⁻¹.

As with electron density prediction, previous works on predicting the DOS can be divided between a grid-based scheme and an atom-centered approach. Focusing on the more recent studies, one such study predicts the DOS through the LDOS for liquid and solid Al using a grid-based approach. The DOS obtained from the predicted LDOS shows very good accuracy, but the DOS of Al presents a smooth shape with very small variations, even from solid to liquid. However, the computational cost of using a grid-based approach can significantly hinder the use of the method for large databases.

Another approach used a significantly different and much more advanced method to represent the atomic structure based on graph neural networks along with an encoder-decoder technique. The method learns similarity between crystalline structures which is then translated into the DOS. One advantage of this technique is probably the lower cost when applying to diverse chemistries. However, it seems focused on crystalline structures, and its application to slightly unrelaxed structures is dependent upon the atomic coordinates being sufficiently similar to any of the DFT-relaxed structures used during training.

FIGS. 19A-19F also include the DFT and ML-DFT VBM and CBM. Due to the use of the DFT eigenvalues for VBM and CBM along with the smeared DOS as reference, the locations of DFT and ML-DFT VBM and CBM may not be at zero-valued DOS. The ML-DFT VBM and CBM values can be in excellent agreement with the reference DFT eigenvalues, as can also be observed from the parity plots between the DFT reference and the predicted ML-DFT values of VBM, CBM, and E_{gap}, in FIGS. 19G-19I, respectively. The mean absolute errors for VBM, CBM and E_{gap} can be 0.069 eV, 0.0191 eV and 0.083 eV, respectively.

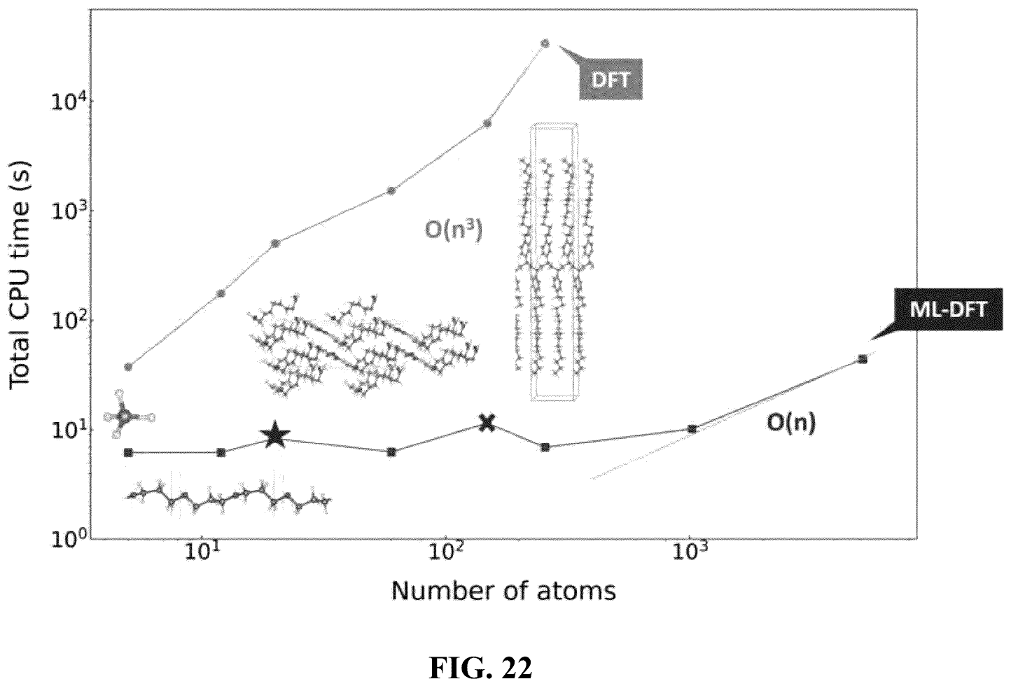

In FIG. 22 the computational cost of DFT is compared with the ML-DFT approach, for various structures of different sizes. Both types of calculations (DFT and ML-DFT) can be performed in serial mode on one core of a Ryzen 9 59000X node. The total CPU time cost of DFT can be significantly higher and can have a cubic dependence on the system size. However, the total time for the electronic structure prediction with the ML-DFT can be orders of magnitude lower than DFT with a linear dependence on system size. The ML-DFT model can depend on the number of element types in the system: the squares represent cases with only carbon and hydrogen; the star is for a polymer chain with three elements (carbon, hydrogen, and oxygen); and the cross represents the case of a polymer crystal with all four elements.

### DISCUSSION

The present invention represents an important step towards a physically-informed ML-based DFT emulator, which can successfully, accurately, and simultaneously reproduce many of the outputs of the KS equation. Following the essence of DFT, material properties can be determined by the descriptors of the structure and the predicted charge density, resulting in an increased accuracy with respect to traditional ML potentials for a fraction of the computational cost of traditional DFT.

To represent the charge density with physically-informed descriptors, GTOs can be employed, which can fully adapt to each individual atom and can be used for any element type. The resulting descriptors can contain physical information of the system in two ways. First, as descriptors of the electronic structure of the system, which can be employed to further predict density-dependent properties. This density-enhanced protocol can be demonstrated for the atomic properties of energy, atomic forces, and stress tensor, and can obtain better performance and more robust models than by using only atomic structure descriptors. Second, the descriptors can be used to directly calculate the partial atomic charges, and can obtain high accuracy when compared to the leading methods in the field. Further applications of these physically-informed descriptors of the charge density for the prediction of other electron density related properties such as the dipole moment or polarizability can be predicted.

The performance and robustness of the ML-DFT approach can allow for a wide range of applications: from electronic structure prediction, to structure search and optimization, while integration with MD codes can allow for emulations of ab initio MD simulations. Large scale dynamical simulations of disordered systems such as liquids or glasses may be performed, which can be challenging for traditional DFT. Generalization of this methodology to more delocalized electron densities, such as in metals, may employ modifications to the type of basis functions used in the decomposition of the charge density. Further modifications may also increase the accuracy and transferability by shifting the present fingerprinting from predefined equations to allow deep learning architectures to search for the best atomic descriptors.

### Methods

### DFT Details

All the reference data calculations can be performed using DFT-MD simulations using the Vienna Ab Initio Simulation Package (VASP). The exchange-correlation functional can be modeled using the Perdew-Burke-Ernzerhof approximation and the ion-electron interaction can be modeled using projector-augmented wave (PAW) potentials. A Monkhorst-Pack grid with a density of 0.025 A⁻¹ can be employed to sample the Brillouin zone. A plane wave basis set with kinetic energy cutoff of 500 eV can be used. The chosen kinetic energy cutoff and k-point sampling can converge the total energy to less than 1 meV per atom. Tkatchenko and Scheffler vdW correction can be included. A Gaussian smearing of 0.1 eV can be used. The MD simulations can be performed in the NVT ensemble, with a time step of 1 fs for the molecules and polymer chains at 300 K. For the polymer crystals, due to the high temperatures reached, a timestep of 0.5 fs can be used. All structures can be thermalized for 500 time steps at their initial temperature (300 or 100 K) and the snapshots can be taken from the subsequent simulations spanning 1 ps for the molecules and polymer chains, and 5 ps for the crystal polymers.

### AGNI Fingerprints

For a given atom i, three different type of AGNI fingerprints can be defined (scalar, vector, and tensor), expressed as the sum over the number of Gaussian functions (k) of width σₖ,

$${S}_{k , i} = {c}_{k} {\sum }_{j = 1}^{N} exp ( \frac{- {R}_{ij}^{2}}{2 {σ}_{k}^{2}} ) {f}_{c} ( {R}_{ij} ) \qquad Equation 10$$

$${V}_{k , i}^{α} = {c}_{k} {\sum }_{j = 1}^{N} \frac{{r}_{ij}^{α}}{{R}_{ij}} exp ( \frac{- {R}_{ij}^{2}}{2 {σ}_{k}^{2}} ) {f}_{c} ( {R}_{ij} ) \qquad Equation 11$$

$${T}_{k , i}^{αβ} = {c}_{k} {\sum }_{j = 1}^{N} \frac{{r}_{ij}^{α} {r}_{ij}^{β}}{{R}_{ij}^{2}} exp ( \frac{- {R}_{ij}^{2}}{2 {σ}_{k}^{2}} ) {f}_{c} ( {R}_{ij} ) \qquad Equation 12$$

where cₖ is the normalization constant defined as

$${( \frac{1}{\sqrt{2 π {σ}_{k}}} )}^{3} ,$$

Rᵢⱼ the distance between atom j and the center atom i, and f_{c}(Rᵢⱼ) a cutoff function defined as

$$0.5 [ cos ( \frac{π ( {R}_{i j}}{{d}_{c}} ) ]$$

for Rᵢⱼ≤d_{c}, and equal to 0 for Rᵢⱼ>d_{c}. α and β represent the x, y, or z components of the radial vector between atoms i and j. In an exemplary embodiment, 18 different Gaussian widths can be employed, on a logarithmic scale (base 10) from 0.5 to 6.0° A, with a cutoff distance of d_{c}=5° A. While Sₖ can be rotation invariant, Vₖ^{α} and Tₖ^{αβ} are not, but can be combined into four rotation invariant expressions, which can be employed as the fingerprints.

### ML-DFT Architecture

In an exemplary embodiment, Keras with Tensorflow backend can be used to implement the ML-DFT. The charge density and DOS models can employ fully connected layers and can be trained using a mini-batch of 30, while the model for the potential energy, atomic force, and stress tensor can use a mini-batch of 100. All models can use random sampling along with Adam optimizer with a learning rate of 0.0001 and momentum vectors β₁=0.9 and β₂=0.999. The mean-squared-error (MSE) can be employed as the objective function during all training.

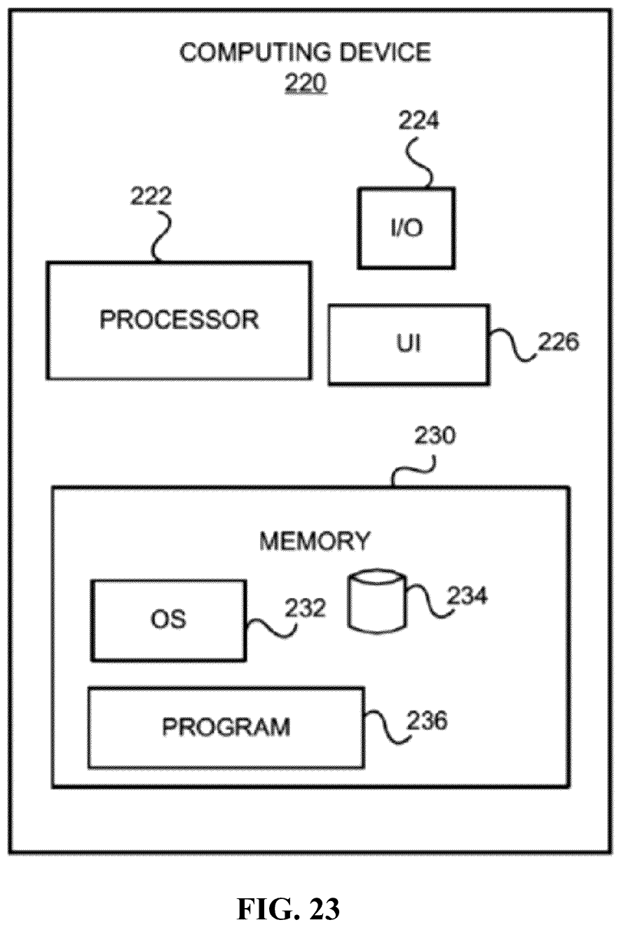

FIG. 23 illustrates an exemplary computing device that can be used to implement the methods (or one or more steps of the methods) disclosed herein. As will be appreciated by one of skill in the art, the computing device 220 can be configured to implement all or some of the features described in relation to the methods disclosed herein. As shown, the computing device 220 may include a processor 222, an input/output (“I/O”) device 224, a memory 230 containing an operating system (“OS”) 232 and a program 236. In certain example implementations, the computing device 220 may be a single server or may be configured as a distributed computer system including multiple servers or computers that interoperate to perform one or more of the processes and functionalities associated with the disclosed embodiments. In some embodiments, computing device 220 may be one or more servers from a serverless or scaling server system. In some embodiments, the computing device 220 may further include a peripheral interface, a transceiver, a mobile network interface in communication with the processor 222, a bus configured to facilitate communication between the various components of the computing device 220, and a power source configured to power one or more components of the computing device 220.

A peripheral interface, for example, may include the hardware, firmware and/or software that enable(s) communication with various peripheral devices, such as media drives (e.g., magnetic disk, solid state, or optical disk drives), other processing devices, or any other input source used in connection with the disclosed technology. In some embodiments, a peripheral interface may include a serial port, a parallel port, a general-purpose input and output (GPIO) port, a game port, a universal serial bus (USB), a micro-USB port, a high definition multimedia interface (HDMI) port, a video port, an audio port, a Bluetooth™ port, a near-field communication (NFC) port, another like communication interface, or any combination thereof.

In some embodiments, a transceiver may be configured to communicate with compatible devices and ID tags when they are within a predetermined range. A transceiver may be compatible with one or more of: radio-frequency identification (RFID), near-field communication (NFC), Bluetooth™, low-energy Bluetooth™ (BLE), WiFi™, ZigBee™, ambient backscatter communications (ABC) protocols or similar technologies.

A mobile network interface may provide access to a cellular network, the Internet, or another wide-area or local area network. In some embodiments, a mobile network interface may include hardware, firmware, and/or software that allow(s) the processor(s) 222 to communicate with other devices via wired or wireless networks, whether local or wide area, private or public, as known in the art. A power source may be configured to provide an appropriate alternating current (AC) or direct current (DC) to power components.

In accordance with certain example implementations of the disclosed technology, the computing device 220 may include one or more storage devices configured to store information used by the processor 222 (or other components) to perform certain functions related to the disclosed embodiments. In one example, the computing device 220 may include the memory 230 that includes instructions to enable the processor 222 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, the computing device 220 may include a memory 230 that includes instructions that, when executed by the processor 222, perform one or more processes consistent with the functionalities disclosed herein. Methods, systems, and articles of manufacture consistent with disclosed embodiments are not limited to separate programs or computers configured to perform dedicated tasks. For example, the computing device 220 may include the memory 230 that may include one or more programs 236 to perform one or more functions of the disclosed embodiments.

In example embodiments of the disclosed technology, the computing device 220 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While the computing device 220 has been described as one form for implementing the techniques described herein, other, functionally equivalent, techniques may be employed. For example, some or all of the functionality implemented via executable instructions may also be implemented using firmware and/or hardware devices such as application specific integrated circuits (ASICs), programmable logic arrays, state machines, etc. Furthermore, other implementations of the computing device 220 may include a greater or lesser number of components than those illustrated.

The processor 222 may include one or more of a microprocessor, microcontroller, digital signal processor, co-processor or the like or combinations thereof capable of executing stored instructions and operating upon stored data. The memory 230 may include, in some implementations, one or more suitable types of memory (e.g., such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs (including, for example, a web browser application, a widget or gadget engine, and or other applications, as necessary), executable instructions and data. In one embodiment, the processing techniques described herein may be implemented as a combination of executable instructions and data stored within the memory 230.

The processor 222 may be one or more known processing devices, such as, but not limited to, a microprocessor from the Pentium™ family manufactured by Intel™ or the Turion™ family manufactured by AMD™. The processor 222 may constitute a single core or multiple core processor that executes parallel processes simultaneously. For example, the processor 222 may be a single core processor that is configured with virtual processing technologies. In certain embodiments, the processor 222 may use logical processors to simultaneously execute and control multiple processes. The processor 222 may implement virtual machine technologies, or other similar known technologies to provide the ability to execute, control, run, manipulate, store, etc. multiple software processes, applications, programs, etc. The processor 222 may also comprise multiple processors, each of which is configured to implement one or more features/steps of the disclosed technology. One of ordinary skill in the art would understand that other types of processor arrangements could be implemented that provide for the capabilities disclosed herein.

The processor 222 may execute one or more programs located remotely from the computing device 220. For example, the computing device 220 may access one or more remote programs that, when executed, perform functions related to disclosed embodiments.

The computing device 220 may also include one or more I/O devices 224 that may comprise one or more user interfaces 226 for receiving signals or input from devices and providing signals or output to one or more devices that allow data to be received and/or transmitted by the computing device 220. For example, the computing device 220 may include interface components, which may provide interfaces to one or more input devices, such as one or more keyboards, mouse devices, touch screens, track pads, trackballs, scroll wheels, digital cameras, microphones, sensors, and the like, that enable the computing device 220 to receive data from a user.

The memory 230 may include one or more memory devices that store data and instructions used to perform one or more features of the disclosed embodiments. The memory 230 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The memory 230 may include software components that, when executed by the processor 222, perform one or more processes consistent with the disclosed embodiments. In some examples, the memory 230 may include a database 234 configured to store various data described herein. For example, the database 234 can be configured to store the software repository or data generated by the repository intent model such as synopses of the computer instructions stored in the software repository, inputs received from a user (e.g., responses to questions or edits made to synopses), or other data that can be used to train the repository intent model.

The computing device 220 may also be communicatively connected to one or more memory devices (e.g., databases) locally or through a network. The remote memory devices may be configured to store information and may be accessed and/or managed by the computing device 220. By way of example, the remote memory devices may be document management systems, Microsoft™ SQL database, SharePoint™ databases, Oracle™ databases, Sybase™ databases, or other relational or non-relational databases. Systems and methods consistent with disclosed embodiments, however, are not limited to separate databases or even to the use of a database.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method comprising:
providing a training data set to a machine learning (ML) system, the training data set indicative of DFT data for a plurality of materials, the DFT data representing atomic configurations for the plurality of materials;
determining a fingerprint for the atomic configuration for each of the plurality of materials;
inputting the fingerprints into the ML system;
generating, with the ML system, electron charge density data for the plurality of materials;
inputting the electron charge density data and the fingerprints into the ML system; and
generating, with the ML system, one or more DFT properties of the plurality of materials.

2. The method of claim 1, wherein the plurality of materials comprises molecules, polymer chains, polymer crystal structures, or combinations thereof.

3. The method of claim 1, wherein the fingerprints are indicative of a structural and/or chemical environment of each of the corresponding material.

4. The method of claim 1, wherein the electronic charge density data comprises one or more Gaussian-type orbitals (“GTOs”).

5. The method of claim 4 further comprising projecting the one or more GTOs onto grid points.

6. The method of claim 1, wherein the one or more DFT properties comprise one or more from the following list: potential energy, atomic force, stress tensor, density of states, valence band maximum, conduction band minimum, and bandgap.

7. The method of claim 1, wherein the plurality of materials comprises a quantity of atoms, wherein a time of prediction for the ML system is approximately linearly dependent on the quantity of atoms.
